(19) United States
(12) Patent Application Publication
(10) Pub. No.: US 2024/0198023 A1
(43) Pub. Date: Jun. 20, 2024

(54) SUCTION AND OXYGEN THERAPY POD HAVING SENSOR TO DETECT CHANGES IN REMOVABLE ACCESSORIES

(71) Applicant: BeaconMedaes LLC, Rock Hill, SC (US)

(72) Inventors: Jay D’Agostino, Coram, NY (US); Greg DelBene, Davidson, NC (US); Rocco Morelli, Lincolnshire, IL (US)

(21) Appl. No.: 18/472,691

(22) Filed: Sep. 22, 2023

Related U.S. Application Data
(60) Provisional application No. 63/476,011, filed on Dec. 19, 2022.

(51) Int. Cl. A61M 16/00; A61M 16/08

(52) U.S. Cl. CPC A61M 16/024; A61M 16/0051; A61M 16/0057; A61M 16/0816; A61M 2205/3306; A61M 2205/3382; A61M 2205/3386; A61M 2205/581; A61M 2205/583

(57) ABSTRACT

A pod including a flow management device disposed within a pod casing, at least one fluidic coupling within the casing that is fluidically connected to the flow management device, the at least one fluidic coupling configured to fluidically connect with a removable accessory insertable within the pod casing, and a sensor configured to detect a change from a first state to a second state of the removable accessory. A pod system, an accessory insertable within the pod, and a method is also provided.

### CROSS-REFERENCE TO RELATED APPLICATIONS

This application claims the benefit of and priority to U.S. Provisional Application No. 63/476,011, filed Dec. 19, 2022, and entitled “Suction and Oxygen Therapy Pod Having Sensor to Detect Changes in Removable Accessories.”

### FIELD OF TECHNOLOGY

The following relates to embodiments of a pod, and more specifically to embodiments of a pod having a sensor to detect changes in a removable accessory of the pod, a pod system, and a method thereof.

### BACKGROUND

Conventional Suction and Oxygen Therapy (SOT) in medical facilities include flow regulators for medical gas and suction regulators that are connected to existing ports located on a wall of a room in a medical facility. Tubes connect to the flow regulators and suction regulators and then connect to the patient or medical equipment used to treat a patient. The flow regulators often have canisters that store a medium that is added or depleted from the canister during use of the SOT devices.

### SUMMARY

An aspect relates to a pod comprising a flow management device disposed within a pod casing, at least one fluidic coupling within the casing that is fluidically connected to the flow management device, the at least one fluidic coupling configured to fluidically connect with a removable accessory insertable within the pod casing, and a sensor configured to detect a change from a first state to a second state of the removable accessory.

In an exemplary embodiment, the first state of the removable accessory is a level of a medium inside a canister of the removable accessory that is below a predefined level, and the second state of the removable accessory is the level of the medium inside the canister exceeding the predefined level. Alternatively, the first state of the removable accessory is a level of a medium inside a canister of the removable accessory that is above a predefined level, and the second state of the canister is the level of the medium inside the canister falling below the predefined level.

When the sensor detects the change from the first state to the second state, the sensor sends a signal to a control interface of the pod for performing at least one function. The function can be an alarm or notification or can be an automatic response carried out without clinician involvement. The alarm response can be notifying a clinician with at least one of an audible notification, a visual notification on a graphical user interface, an indicator, a transmission to a remote computer, or a combination thereof. The at least one automatic response function is returning the removable accessory to the first state, an injection of a solution into a canister of the removable accessory storing a medium filling a canister of the removable accessory with an additional medium, and/or an evacuation of a canister of the removable accessory to remove a medium from the canister.

In an exemplary embodiment, the sensor is an optical sensor housed within the pod.

When a removable accessory is inserted within the pod casing, the at least one fluidic coupling mates with at least one corresponding fluidic coupling of the removable accessory within the casing, and fluid managed by the flow management device flows through the removable accessory and in or out of an outlet of the removable accessory that is fluidically connected to the at least one corresponding fluidic coupling. The pod includes an accessory receiving mechanism located within the pod casing, the accessory receiving mechanism facilitating an insertion and fluidic coupling of the accessory within the pod casing.

Another aspect relates to a system comprising a pod comprising a flow management device disposed within a pod casing, at least one fluidic coupling within the casing that is fluidically connected to the flow management device, the at least one fluidic coupling configured to fluidically connect with a removable accessory insertable within the pod casing, a removable accessory for use with the pod, the removable accessory comprising: a body portion having a first side and a second side, at least one fluidic coupling disposed on the body portion, and a canister operably attached to the body portion, configured to store a medium, a sensor configured to detect a change from a first state to a second state of the canister; and a control interface electrically connected to the sensor and configured to receive a signal from the sensor indicating the change.

In an exemplary embodiment, the system can also include a pump fluidically connected to an interior of the canister, the pump electrically connected to the control interface and configured to inject a solution into the interior of the canister in response to a signal from the control interface based on the sensor detecting the change. The system can also include a vacuum pump fluidically connected to an interior of the canister, the vacuum electrically connected to the control interface and configured to evacuate the interior of the canister in response to a signal from the control interface based on the sensor detecting the change. The system can also include a pump fluidically connected to an interior of the canister, the pump electrically connected to the control interface and configured to add an additional medium to the interior of the canister in response to a signal from the control interface based on the sensor detecting the change.

Another aspect relates to a method comprising: monitoring a removable accessory inserted into a pod, detecting a change from a first state to a second state of the removable accessory, by a sensor of the pod, and performing a function by a control interface of the pod, in response to the detecting.

The foregoing and other features of construction and operation will be more readily understood and fully appreciated from the following detailed disclosure, taken in conjunction with accompanying drawings.

### BRIEF DESCRIPTION OF THE DRAWINGS

Some of the embodiments will be described in detail, with reference to the following FIGURES, wherein like designations denote like members, wherein:

FIG. 1 depicts a schematic view of a rough-in assembly having connections to a plurality of sources, in accordance with embodiments of the present invention;

FIG. 2 depicts a schematic view of pods insertable into the rough-in assembly of FIG. 1, in accordance with embodiments of the present invention;

FIG. 3A depicts a schematic view of a pod inserted into the rough-in assembly to allow access to a first source;

FIG. 3B depicts a schematic view of a pod inserted into the rough-in assembly to allow access to a second source;

FIG. 3C depicts a schematic view of a pod inserted into the rough-in assembly to allow access to a third source;

FIG. 4 depicts a schematic view of a pod inserted within the rough-in assembly, in accordance with embodiments of the present invention;

FIG. 5A depicts a schematic view of a backside of a first pod, in accordance with embodiments of the present invention;

FIG. 5B depicts a schematic view of a backside of a second pod, in accordance with embodiments of the present invention;

FIG. 5C depicts a schematic view of a backside of a third pod, in accordance with embodiments of the present invention;

FIG. 6 depicts three removable accessories that are each capable of being inserted into pod, in accordance with embodiments of the present invention;

FIG. 7 depicts a schematic view of a pod without an accessory inserted therein, in accordance with embodiments of the present invention;

FIG. 8A depicts a first side of an accessory, in accordance with embodiments of the present invention;

FIG. 8B depicts a second side of the accessory, in accordance with embodiments of the present invention;

FIG. 9 depicts a schematic view of the pod with an accessory inserted therein, in accordance with embodiments of the present invention;

FIG. 10A depicts a first embodiment of a first state of a removable accessory, in accordance with embodiments of the present invention;

FIG. 10B depicts a first embodiment of a second state of the removable accessory, changed from the first state shown in FIG. 10A, in accordance with embodiments of the present invention;

FIG. 10C depicts a second embodiment of a first state of a removable accessory, in accordance with embodiments of the present invention;

FIG. 10D depicts a second embodiment of a second state of the removable accessory, changed from the first state shown in FIG. 10C, in accordance with embodiments of the present invention;

FIG. 11 depicts a schematic view of the pod with an accessory inserted therein and a cover covering the accessory, in accordance with embodiments of the present invention;

FIG. 12 depicts a pod system incorporated into various objected in an environment, in accordance with embodiments of the present invention;

FIG. 13 depicts a front view of a pod system according to an exemplary embodiment of the present invention;

FIG. 14 depicts a perspective view of the pod system shown in FIG. 13, with a cover 60 installed onto the pods, in accordance with embodiments of the present invention;

FIG. 15 depicts a perspective view of another embodiment of a pod system, in accordance with embodiments of the present invention;

FIG. 16 depicts a front view of the pod system shown in FIG. 15, in accordance with embodiments of the present invention;

FIG. 17 depicts a perspective view of three pods, according to an exemplary embodiment of the present invention;

FIG. 18A depicts a perspective view of an embodiment of the pod without an accessory inserted therein, in accordance with embodiments of the present invention;

FIG. 18B depicts a perspective view of pod removed from the pod casing, in accordance with embodiments of the present invention;

FIG. 19A depicts a perspective view of an embodiment of the pod without an accessory inserted therein, in accordance with embodiments of the present invention;

FIG. 19B depicts a perspective view of pod removed from the pod casing, in accordance with embodiments of the present invention;

FIG. 20 depicts a bottom, perspective view of an accessory and a pod before insertion of the accessory into the pod, in accordance with an exemplary embodiment of the present invention;

FIG. 21 depicts a perspective view of an accessory and a pod before insertion of the accessory into the pod, in accordance with an exemplary embodiment of the present invention;

FIG. 22 depicts a connection between an accessory and a pod, in accordance with an exemplary embodiment of the present invention;

FIG. 23 depicts a first type of accessory, in accordance with an exemplary embodiment of the present invention;

FIG. 24 depicts a second type of accessory, in accordance with an exemplary embodiment of the present invention;

FIG. 25 depicts a third type of accessory, in accordance with an exemplary embodiment of the present invention;

FIG. 26 depicts a schematic view of a fluid evacuation system that drains to a collection vessel or main plumbing system, in accordance with embodiments of the present invention;

FIG. 27 depicts a schematic view of a fluid filling system, in accordance with embodiments of the present invention; and

FIG. 28 depicts a schematic view of a fluid injection system that injects a solution into the pod, in accordance with embodiments of the present invention.

### DETAILED DESCRIPTION

A detailed description of the hereinafter described embodiments of the disclosed apparatus and method are presented herein by way of exemplification and not limitation with reference to the Figures. Although certain embodiments are shown and described in detail, it should be understood that various changes and modifications may be made without departing from the scope of the appended claims. The scope of the present disclosure will in no way be limited to the number of constituting components, the materials thereof, the shapes thereof, the relative arrangement thereof, etc., and are disclosed simply as an example of embodiments of the present disclosure.

As a preface to the detailed description, it should be noted that, as used in this specification and the appended claims, the singular forms “a”, “an” and “the” include plural referents, unless the context clearly dictates otherwise.

In brief overview, SOT solutions include medical gas flow regulators and suction regulators that attach to existing medical gas outlets on a wall of a hospital room. Conventional SOT devices protrude from the wall because the flow regulator mechanism is located external from the wall, which leads to several problems. Incidental contact with the SOT devices extending outward from the wall can damage the devices. Clinicians remove the SOT devices from the wall in a hospital room to use in another room and thus the current hospital room is left without a critical SOT device. The tubes attached to conventional SOT devices often get disconnected which means a supply of gas can enter the room without anyone knowing.

Further, conventional flow regulators and suction units used in SOT have canisters attached to the devices which are also located external to the wall. The canisters store or otherwise receive medium, such as water, medicine, solutions, biological waste, etc. The medium is depleted or filled with additional medium over time, which requires action on behalf of the attending clinician. If the attending clinician is treating another patient and/or not present in the hospital room, the attending clinician is unaware of the need to take action to replace the canister, add additional medium, evacuate the canister, or otherwise attend to the canister. As time goes on without the canister receiving attention, the performance of the SOT device can decline, or worse, the patient is receiving less than ideal treatment and care.

Embodiments of the present invention utilize a pod sensor for detecting changes in the state of a removable accessory (e.g. canister), and either alert the attending clinician to the need to attend to the removable accessory, or send a signal to a control interface for automatically taking needed action. The pod sensor is part of a pod that may be part of a pod system that is recessed inside a wall (or a headwall of a bed unit). Each pod of the pod system has a flow management device (e.g. oxygen, medical air, vacuum, etc.) that fits within a rough-in box behind the wall and a sensor to detect changes in a state of the removable accessory associated with the pod. If the sensor detects a change in state of the removable accessory, the sensor sends a signal to a control interface of the pod for performing at least one function, such as alerting a clinician, triggering a notification, returning the removable accessory to a previous state, injecting a medium inside the removable accessory, or evacuating the removable accessory.

The fluidic connections of the pod system are made within the recess of the wall. The pods have a built-in flow management device (e.g. electronic solenoid valve) that regulates the flow of gas in both directions through the pod from a source. Each pod is recessed into the wall and includes a control unit with a user interface that allows a clinician to adjust the flow/suction using the user interface. The pods also include a portion that allows a mechanical connection to a pod frame, with a port that allows for an attachment of tubing to go from the pod to the patient. Various accessories can be removably attached to the pod, establishing fluidic communication with a fluid source located remotely; the pod sensor monitors the accessories. A casing of the pod includes structure for easy attachment of accessories to the pod, which can be slid into the pod shell and locked into place. Thus, conventional SOT devices that attach to the wall are replaced by the pod system disclosed herein which includes pods recessed within a wall of a hospital room, the connections of which are secure within the pod in the wall, while also having a sensor to monitor the accessory.

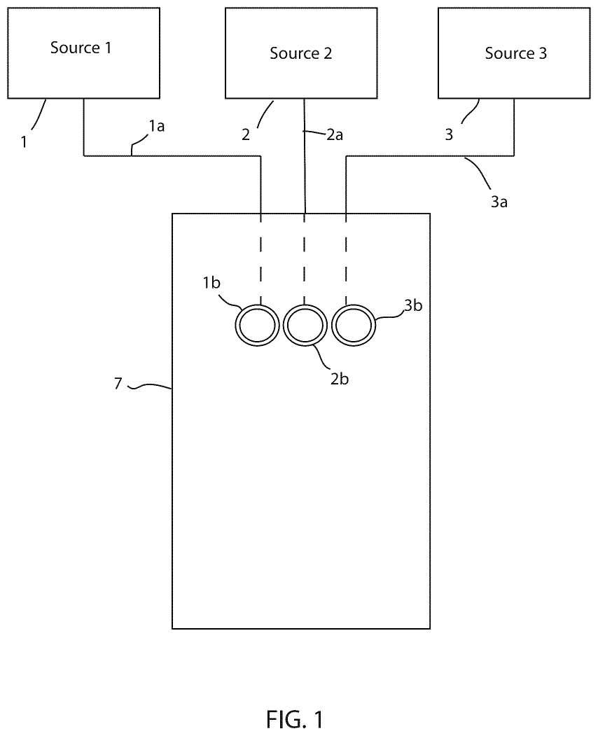

Referring now to the drawings, FIG. 1 depicts a schematic view of a rough-in assembly 7 having connections to a plurality of sources 1, 2, 3, in accordance with embodiments of the present invention. Rough-in assembly 7 is located behind a finished surface, such as a wall of a hospital room, and is configured to be attached to one or more structures located behind the finished surface, such as metal or wood wall construction elements. Rough-in assembly 7 is a receptacle safe for in-wall or covered applications that has an open face to accommodate one or more pods of a pod system, as described in greater detail infra. The rough-in assembly 7 is sized and dimensioned to allow a pod to fit therein. In an exemplary embodiment, a depth of the rough-in assembly 7 can be large enough so that a pod fits entirely within the rough-in assembly 7 so that a front surface of the pod can be flush or recessed with a finished surface. In another embodiment, the depth of the rough-in assembly 7 can be reduced such that only a portion of the pod fits within the rough-in assembly 7 so that a front surface of the pod protrudes slightly from the finished surface.

The rough-in assembly 7 accommodates connections to sources 1, 2, 3 located remote from the rough-in assembly 7. For instance, rough-in assembly 7 safely and securely accommodates fluidic couplings 1b, 2b, 3b of the sources 1, 2, 3 within the rough-in assembly 7. In the illustrated embodiment, supply lines 1a, 2a, 3a associated with a first source 1, a second source 2, and a third source 3, respectively enter the rough-in assembly 7 through a back wall of the rough-in assembly 7; however, the supply lines 1a, 2a, 3a may be directed through any surface of the rough-in assembly 7. The supply lines 1a, 2a, 3a pass through openings either pre-formed or created in the field, such as by removing knockouts on the rough-in assembly 7. The fluidic couplings 1b, 2b, 3b are devices configured to couple or connect two fluidic channels together. A type and/or size of the fluidic coupling 1b, 2b, 3b depends on the source 1, 2, 3. Examples of fluidic couplings 1b, 2b, 3b include a fitting, a connector, an adapter, check valves, hose barbs, elbows, quick-connect, etc. After installation of the rough-in assembly 7 and fluidic couplings 1b, 2b, 3b within the rough-in assembly 7, access to any of the sources 1, 2, 3 is possible, depending on a type of pod inserted within the rough-in assembly 7, as described in greater detail infra.

While three sources are depicted in FIG. 1, less than three or more than three sources can be roughed in to rough-in assembly 7. The sources 1, 2, 3 can be tanks, reservoirs, vessels, containers, and the like, storing a gas, fluid, or mixture of gases, with a mechanism for delivering the gas, fluid, or mixture from their remote location via supply lines 1a, 2a, 3a. A non-exhaustive list of the types of sources 1, 2, 3 includes medical air (Med Air), carbon dioxide (CO2), helium (He), nitrogen (N2), nitrous oxide (N20), oxygen (O2), oxygen/carbon dioxide mixture (O2/CO2 n %, n is % of CO2), medical-surgical vacuum (Med Vac), waste anesthetic gas disposal (WAGD), non-medical air (Level 3 gas-powered device), laboratory air, laboratory vacuum, instrument air, and other mixtures having a ratio of Gas A/Gas B in percentage. Further, reference to SOT is a term intended to be inclusive of gases that contain oxygen and that do not necessarily include oxygen.

In FIG. 1, the first source 1 is a source of oxygen, the second source 2 is a source of medical air, and the third source 3 is a vacuum source. The first source 1 is accessible via the fluidic coupling 1b within the rough-in assembly 7 via supply line 1a, which is connected to the first source 1 at one end and connected to the fluidic coupling 1b at the other end. Similarly, the second source 2 is accessible via the fluidic coupling 2b within the rough-in assembly 7 via supply line 2a, which is connected to the second source 2 at one end and connected to the fluidic coupling 2b at the other end, and the third source 3 is accessible via the fluidic coupling 3b within the rough-in assembly 7 via supply line 3a, which is connected to the third source 3 at one end and connected to the fluidic coupling 3b at the other end. Supply lines 1a, 2a, 3a are located behind a finished surface and can have various lengths and intermediate fittings to accommodate runs from the source to the rough-in assembly 7. The supply lines 1a, 2a, 3a are terminated by the fluidic couplings 1b, 2b, 3b inside the rough-in assembly 7. Located at least partially within a casing 10 of the pod 100. Alternatively, the supply lines 1a, 2a, 3a are terminated outside of the rough-in assembly 7, behind the finished surface, with an intermediate connection line completing the connection from the ends of the supply line 1a, 2a, 3a to the fluidic couplings 1b, 2b, 3b within the rough-in assembly 7; the intermediate connection line can be a flexible tubing that provides flexibility to connect the sources to rough-in assembly 7 in certain construction environments where existing piping from the sources is connected to a new installation of the rough-in assembly 7.

Because the rough-in assembly 7 houses fluidic connections to multiple sources 1, 2, 3, any of the sources 1, 2, 3 can be accessed at the location of the rough-in assembly 7. The fluidic couplings 1b, 2b, 3b within the rough-in assembly 7 are discrete and separate from each other so that a connection can be made to one fluidic coupling only and not the others; thus, only gas from the source associated with the fluidic coupling which has a mated connection flows through the pod system, while the other fluidic couplings without a mated connection prevent a flow of the gas from the sources into a room environment. Which source 1, 2, 3 is accessed depends on which pod is inserted into the rough-in assembly 7.

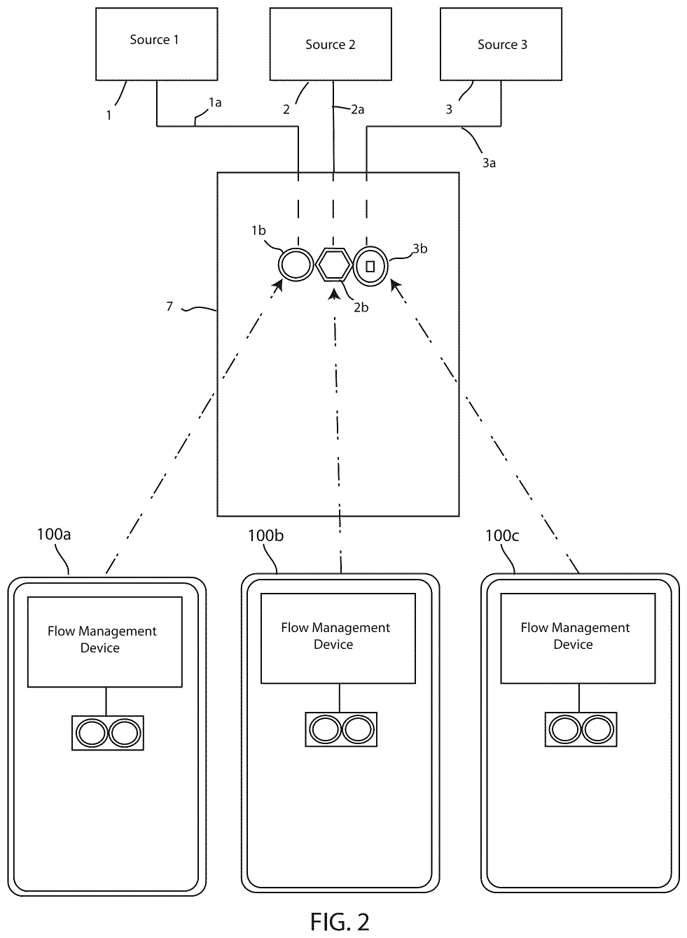

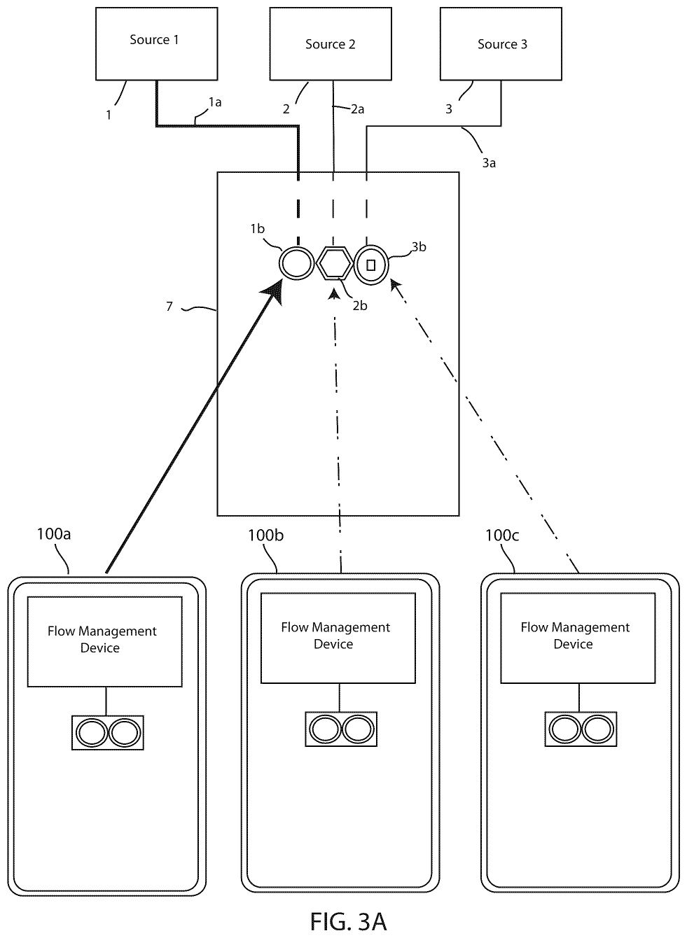

FIG. 2 depicts a schematic view of pods insertable into the rough-in assembly 7 of FIG. 1, in accordance with embodiments of the present invention. Each pod is specific to a type of gas associated with a source 1, 2, 3. For example, pod 100a is specifically designed for managing a flow of gas associated with source 1, pod 100b is specifically designed for managing a flow of gas associated with source 2, and pod 100c is specifically designed for managing a flow of gas associated with source 3. If access to source 1 (e.g. oxygen) is desired, then pod 100a can be inserted into rough-assembly 7, as shown in FIG. 3A. If access to source 2 (e.g. medical air) is desired, then pod 100b can be inserted into rough-assembly 7, as shown in FIG. 3B. If access to source 3 (e.g. vacuum) is desired, then pod 100c can be inserted into rough-assembly 7, as shown in FIG. 3C. Each pod 100a. 100b, 100c is removably inserted into the rough-in assembly 7 so that the pod 100a can be removed and swapped with a different pod 100b or 100c, at a same location within the room environment. Because the pods can be swapped out, a clinician can access different types of gases at the same location in the wall, for example, which makes the hospital room more flexible to meet various needs of a medical facility.

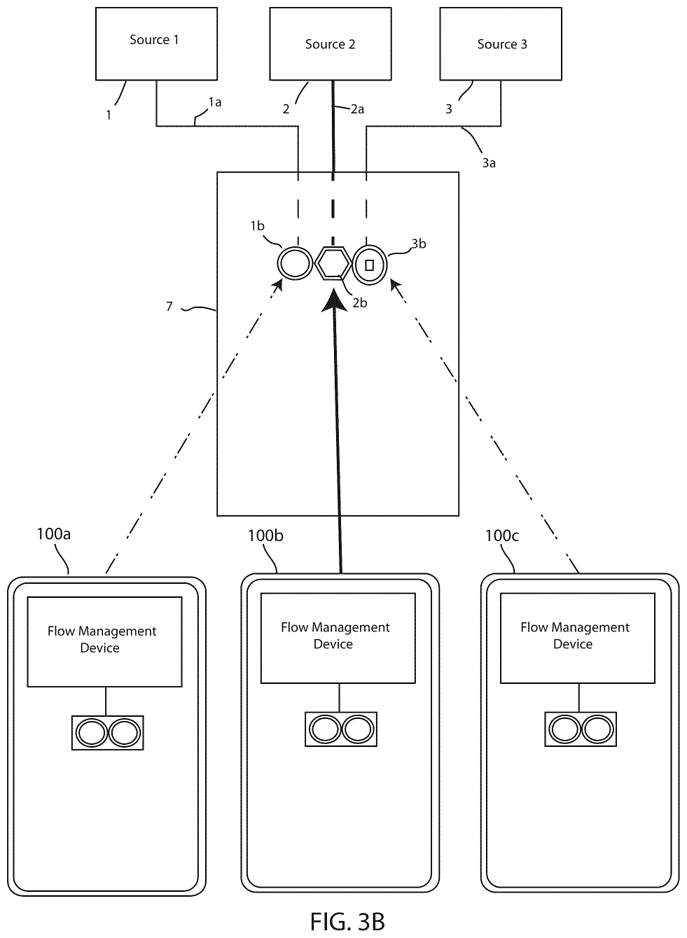

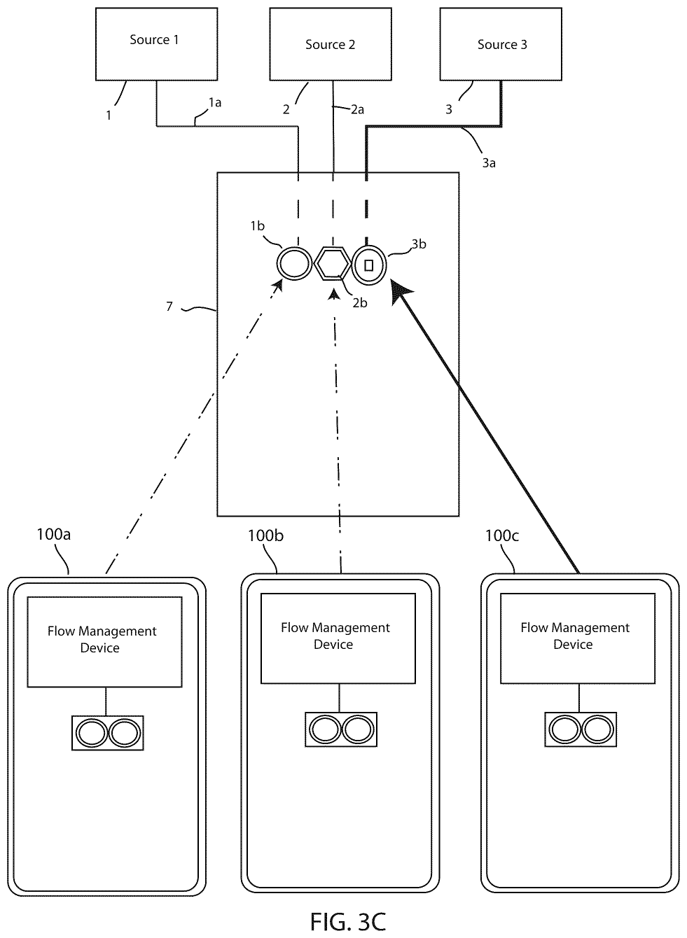

FIG. 4 shows a schematic view of a pod 100 inserted within the rough-in assembly 7, in accordance with embodiments of the present invention. As shown, pod 100 fits within an interior of the rough-in assembly 7; pod 100 could be pod 100a, 100b, 100c, or any pod having a specific type of flow management for use with a specific source. As a function of inserting the pod 100 into the rough-in assembly 7, a fluidic coupling(s) on the back of the pod 100 mates with one of the fluidic couplings 1b, 2b, or 3b that corresponds to the fluidic coupling on the pod 100. FIGS. 5A-5C depict a schematic view of a backside of the pods 100a, 100b, 100c, respectively, in accordance with embodiments of the present invention. The pods 100a, 100b, 100c include a fluidic coupling 11a, 11b, 11c on a back surface 12 of the pod that is designed to mate with one of the fluidic couplings 1a, 1b, 1c. The fluidic couplings 11a, 11b, 11c are designed to mate with one of the fluidic couplings 1a, 1b, 1c based on one or more different structural configurations and/or industry standardized coupling connections. For instance, the shape of the couplings, the location of the couplings, a pin index of the couplings, a diameter of the couplings, etc. can be used to control which couplings 11a, 11b, 11c of the pod can be coupled to the couplings 1a, 1b, 1c of the rough-in assembly 7. In an exemplary embodiment shown in FIGS. 5A-5C, the fluidic couplings 11a, 11b, 11c each have a unique shape (shown schematically) that correspond to a unique shape of the fluidic couplings 1b, 2b, 3b (shown schematically) to indicate that one type of fluidic coupling of the pod is configured to mate with one type of fluidic coupling within the rough-in assembly 7, associated with a specific source of gas.

The mated connected between fluidic coupling 1b, 2b, 2c and 11a, 11b, 11c of the pod allows a flow of gas to a flow management device 20 of the pod 100, which is located within a casing 10 of the pod 100. The flow management device 20 is configured to manage, regulate, or otherwise control a flow of gas through the pod 100, coming from one of the plurality of sources 1, 2, 3. Examples of the flow management device 20 are a flow meter or flow regulator that includes at least one electronic valve for managing a flow of gas through the pod 100. The specific design and construction of the flow management device 20 depends on which type of gas the pod is designed to regulate. For instance, a pod may include a type of flow management device for managing the flow of oxygen, and would also include a fluidic coupling on the back of the pod for mating with the fluidic coupling 1b associated with the first source 1. A pod may include a type of flow management device for managing the flow of medical air, and would also include a fluidic coupling on the back of the pod for mating with the fluidic coupling 2b associated with the second source 2. A pod may include a type of flow management device for managing a vacuum for suction through pod, and would also include a fluidic coupling on the back of the pod for mating with the fluidic coupling 3b associated with the third source 3. Examples of flow management devices include an electronic needle valve and controller, and an integrated mass flow control valve.

A control interface 25 is electrically coupled to the flow management device 20 for controlling at least one function of the flow management device 20. For instance, the control interface 25 includes a controller and a display, and may utilize various input methods such as a touch screen, button interface, and/or rotary dial that allows clinicians to input commands to the flow management device 20 via touch or button press. The control interface 25 sends instructions/commands to the flow management device 20 to perform at least one function, such as maintain a certain flow rate, increase a flow rate, decrease a flow rate, etc. In this way, a clinician can conveniently control the flow management device 20 via the control interface 25 to treat a patient with SOT as required.

Managed or regulated gas flows between the flow management device 20 and at least one fluidic coupling 30 located at least partially within the casing 10 of the pod 100. The fluidic coupling 30 is fluidically connected to the flow management device 20, and, when the pod 100 is inserted within the rough-in assembly 7, the fluidic coupling 30 is also fluidically connected to the supply line and source specific to the pod 100. By operation of the flow management device 20, gas flows from the source through the supply line and through the flow management device 20 to the coupling 30 in a controller manner. The at least one fluidic coupling 30 is a fitting, a connector, an adapter, check valves, hose barbs, elbows, quick-connect, etc. that is configured to mate with a fluidic coupling an accessory insertable within the pod 100. The accessory insertable within the pod includes an outlet though which the regulated gas can flow in or out of the accessory and to the patient via tubing connected to the outlet.

The control interface 25 of pod 100 may generally comprise a processor, an input device coupled to the processor, an output device coupled to the processor, and memory devices each coupled to the processor. The input device, output device and memory devices may each be coupled to the processor via a bus. Processor may perform computations and control the functions of the pod system, including executing instructions included in computer code for the tools and programs capable of implementing a method for detecting a change in state of an accessory insertable within a pod and performing at least one function in response thereto, wherein the instructions of the computer code may be executed by processor via memory device. The computer code may include software program instructions that may implement one or more algorithms for implementing methods and functions of the pod system, as described in detail above. The processor executes the computer code. Processor may include a single processing unit locally resided within the pod housing, or may also be distributed across one or more processing units in one or more locations (e.g., on a client and server).

The memory device of the control interface 25 may include input data that includes any inputs required by the computer code. The output device displays output from the computer code. The memory devices may be used as a computer usable storage medium (or program storage device) having a computer-readable program embodied therein and/or having other data stored therein, wherein the computer-readable program comprises the computer code. Generally, a computer program product (or, alternatively, an article of manufacture) of the computer system may comprise said computer usable storage medium (or said program storage device).

Memory devices of the control interface 25 include any known computer-readable storage medium. In one embodiment, cache memory elements of memory devices may provide temporary storage of at least some program code in order to reduce the number of times code must be retrieved from bulk storage while instructions of the computer code are executed. Moreover, similar to processor, memory devices may reside at a single physical location, including one or more types of data storage, or be distributed across a plurality of physical systems in various forms. Further, memory devices can include data distributed across, for example, a local area network (LAN) or a wide area network (WAN). Further, memory devices may include an operating system.

Pod 100 also includes a sensor 40 electrically coupled to the control interface 25. The sensor 40 is configured to detect a change in a state of a removable accessory inserted within pod 100. The sensor 40 transmits signals to the control interface 25 for performing at least one function, such as alerting a clinician, triggering a notification, returning the removable accessory to a previous state, injecting a medium inside the removable accessory, or evacuating the removable accessory.

Sensor 40 is a contact-less sensor that does not need to physically contact a surface of an object being measured. In an exemplary embodiment, the sensor 40 is an optical sensor, such as a photoconductive device, housed within the pod 100 for detecting various changes to the removable accessory. The optical sensor may include a receiver and a transmitter housed together, positioned on one side of the pod 100. The transmitter transmits or otherwise projects light from a light source, such as a light emitting diode, of the optical sensor, onto the object to be detected (e.g. medium within a canister of accessory). The object to be detected reflects light back towards the optical sensor, which is detected by the receiver of the optical sensor. The amount of light received by the receiver is translated into electrical signals which are output to the control interface 25, or processed by a processor of the sensor. In another embodiment, the sensor 40 is an optical sensor having a transmitter on one side of the pod 100 and a received on the opposite side of the pod 100. The transmitter transmits or otherwise projects a light beam from a light source, such as a light emitting diode, of the optical sensor, through the object to be detected (e.g. canister of accessory). The object to be detected (e.g. medium within a canister) interrupts the light beam in areas where the object is present, and where the object is not present, the light beam continues to the receiver for detection. The amount of light received by the receiver is translated into electrical signals which are output to the control interface 25, or processed by a processor of the sensor. Various optical sensor devices and optical sensor technologies can be used to detect changes in the accessory placed within the pod.

The sensor 40 can be positioned anywhere within the pod 100 provided that the sensor 40 can monitor the accessory inserted within the pod 100, and more particularly, a canister storing contents of the accessory. For instance, the sensor 40 can be positioned on the right wall or the left wall of the pod casing, next to the removable accessory. Optionally, more than one sensor 40 can be located within the pod 100 to detect and/or monitor the accessory from more than one side, angle, perspective, etc. The measurements from the second sensor can be used to confirm a result from the first sensor, for example. One or more sensors 40 can also be used to sense/detect different aspects, metrics, characteristics, etc. of the accessory; the second sensor 40 is shown in dashed lines in FIG. 4. For example, one sensor can be an optical sensor to detect a fluid level within a canister of the accessory while the second sensor can be a temperature sensor to detect a temperature or changes in temperature of a fluid contained within the canister of the accessory. The sensor 40 and the sensor 40 shown in dotted lines can also represent a receiver and transmitter oppositely positioned within the pod 100. Various positions and location of the sensor 40 within the pod 100 can be achieved and may depend on the size, purpose, and design of the accessory. The sensor 40 may be built-in to the flow management device or may be a separate component disposed within an interior of the pod 100.

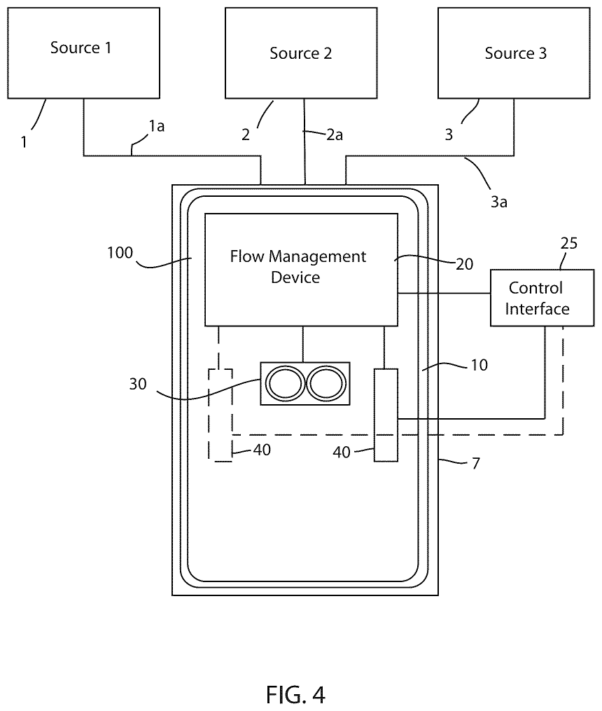

As will now be described with reference to FIG. 6, an accessory can be insertable within the pod 100. The accessory is configured to fluidically and mechanically connect to the fluidic coupling 30 built into the pod 100 so that the gas flows through the accessory and out to the patient. One accessory is insertable into the pod 100 at a given time, but there are many different accessories that can be used with pod 100. The accessory resides within the pod casing 10 when inserted or otherwise attached to the pod 100, and is removable from the pod 100 so that a new accessory can be inserted into the pod 100. FIG. 6 shows three different, removable accessories 5, 6, 7 that are each capable of being inserted into pod 100 but many different accessories can be designed to fit within pod 100. Each accessory 5, 6, 7 includes at least one fluidic coupling 5a, 6a, 7a configured to mate with fluidic coupling 30 of the pod 30, but each accessory has a different function and/or feature that provides flexibility to the clinician.

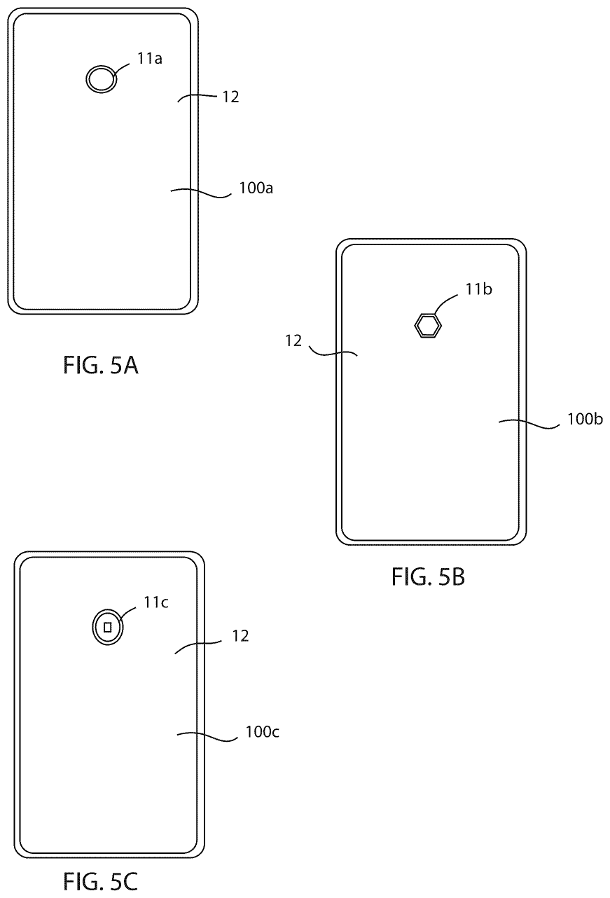

FIG. 7 depicts a schematic view of a pod 100 without an accessory inserted therein, in accordance with embodiments of the present invention. The pod 100, as illustrated in FIG. 7, includes a pod casing 10 that defines an interior region 14 of the pod 100. The casing 10 includes two side walls and back surface (not shown in FIG. 7), and open face to allow insertion of an accessory and installation of the flow management device. The flow management device is located behind the control interface 25 so that a clinician interfaces with the control interface 25 and not the flow management device disposed within the pod 100. The control interface 25 is shown with a plurality of graphical icons and/or buttons for clinician interaction with the pod 100, such as increasing or decreasing a flow rate through the pod 100. The fluidic coupling 30 is disposed within the interior region 14 and is accessible through the open front face of the casing 10 for inserting and removing an accessory.

The sensor 40 is disposed within the interior region 14 of the casing 10. The sensor 40 can be connected to the casing 10 or can be connected to the flow management device disposed above the sensor 40, or can be connected to both the casing 10 and the flow management device. In an embodiment where the sensor 40 is an optical sensor, light can be emitted from the sensor 40 in a direction towards an accessory.

Moreover, the pod 100 includes an accessory receiving mechanism 50 disposed within the casing 10 of the pod 100. The accessory receiving mechanism 50 facilitates an insertion and fluidic coupling of the accessory within the pod casing 10. The accessory receiving mechanism 50 includes a first receiving structure 51 on a first side of the pod casing 10 and a second receiving structure 52 on a second side opposite the first side of the pod casing 10. The first receiving structure 51 and the second receiving structure 52 extend towards the rear wall of the casing 10, starting proximate or at the front of the casing 10. In an exemplary embodiment, the first receiving structure 51 and the second receiving structure 52 are each a lip that protrudes from a housing/enclosure of the sensor 40 that guides the accessory into the casing 10 and into proper mating position with the fluidic coupling 30 of the pod, while supporting, at least partially, the weight of the accessory when inserted and mated within the pod 100. The accessory receiving mechanism 50 is positioned within the pod 100 at a height within the casing 10 to align a fluidic coupling, such as fluidic couplings 5a, 6a, 7a of accessories 5, 6, 7 within the fluidic coupling 30 disposed within the pod casing 10. Thus, the location of the accessory receiving mechanism 50 within the pod 100 can vary across different pod designs and dimensions.

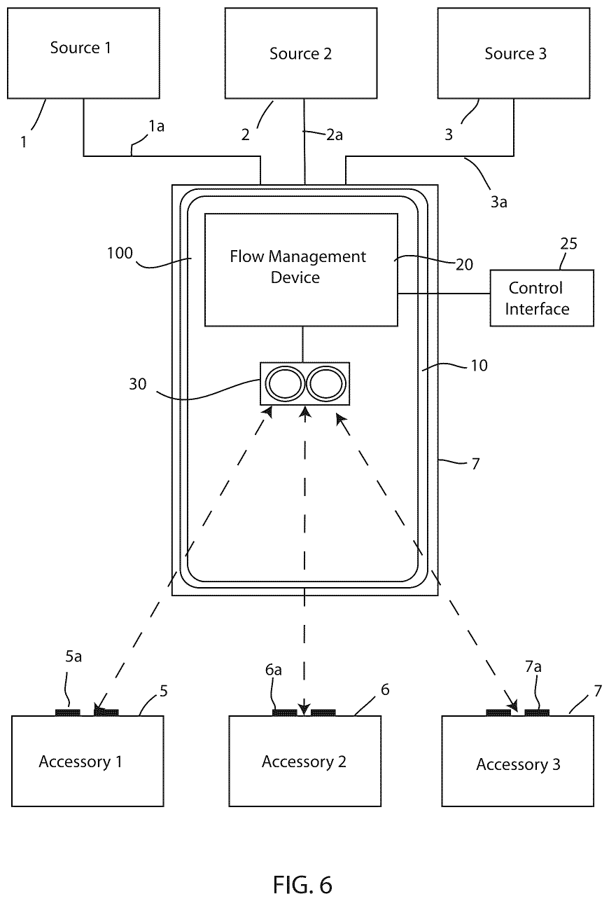

FIGS. 8A-8B depict a schematic view of accessory 70, in accordance with embodiments of the present invention. Accessory 70 could be accessory 5, accessory 6, accessory 7, or any accessory having a specific function or structure, and includes a pod engagement mechanism 76 that universally cooperates with accessory receiving mechanisms 50 of pod 100. The removable accessory 70 includes a body portion 75 having a first side 70a and a second side 70b. The body portion 75 may be a lid or cover that is a solid piece of material capable of being machined or manufactured to include coupling 71 and an outlet 73. The accessory 70 includes a pod engagement mechanism 76 disposed on the body portion 75. The pod engagement mechanism76 includes a first engagement structure at a first location on the body portion 75 and a second engagement structure at a second location of the body portion, opposite the first location. The first engagement structure and the second engagement structure of the pod engagement mechanism 76 is a protrusion or other lip or extension of the body portion 75 that is configured to cooperate with the accessory receiving mechanism 50 of the pod 100.

Moreover, the accessory 70 incudes a canister 74 operably attached to the body portion 75. The canister 74 may be removably attached to the body portion 75 so that the canister 74 of the accessory 70 is further customizable. For instance, the canister 74 may be threadably attached to the body portion 75 so that the canister 74 is easily removed and replaced with a canister 74 of different size or function. The canister 74 may be removably attached to the lower side of the body portion 75 and can be twisted on or off to replace the canister with a new canister of same or different shape/type. The canister 74 is a container or storage device that has an interior space that allows a flow of gas between the fluidic coupling 71 that mates with the fluidic coupling 30 of the pod 100 and the outlet 73. The interior space of the body portion 74 may store a fluid, such as water, for humidifying the gas flowing through the accessory. The interior space of the body portion 74 may also store waste fluid/material drawn into the outlet 73 of the accessory 70.

FIG. 8A depicts the first side 70a of the accessory 70, and FIG. 8B depicts the second side 70b of the accessory 70. The first side 70a of the accessory 70 faces toward a rear wall of the pod 100 when the accessory 70 is removably inserted within the pod casing 10. Fluidic couplings 71 are disposed on the body portion 75 at the first side 70a of the accessory 70 and are configured to mate with the fluidic coupling 30 of the pod 100 when the accessory 70 is removably inserted into the pod 100. While two fluidic couplings 71 are shown in FIG. 8A, there may be a single fluidic coupling that mates with a single fluidic coupling 30 inside the pod 100. Further, while the couplings are depicted as extending from the side of the accessory, the couplings could be made from the top of the accessory as well. The mating of the fluidic couplings 71, 30 establish fluidic communication between the flow management device 20 of the pod 100 and the accessory 70. For instance, at least one fluidic coupling 71 is configured to be fluidically coupled to a flow management device 20 of the pod 100, as a function of the removable accessory 70 being inserted within the pod casing 10 of the pod 100.

The second side 70b of the accessory 70 faces away from a rear wall of the pod 100 when the accessory 70 is removably inserted within the pod casing 10. An outlet 73 is disposed on the body portion 75 at the second side 70b of the accessory 70 and is configured to be accessible by a clinician when the accessory 70 is removably inserted into the pod 100, for attaching tubing that facilitates gas flow between the pod and the patient/patient device. While one outlet 73 is shown in FIG. 8B, there may be a more than outlet disposed on the accessory 70. The outlet 73 is in fluidic communication with the flow management device 20 of the pod 100. The fluid communication may be a dedicated flow path from the fluidic coupling 71 to the outlet 73, or may pass generally through the interior space of the canister 74.

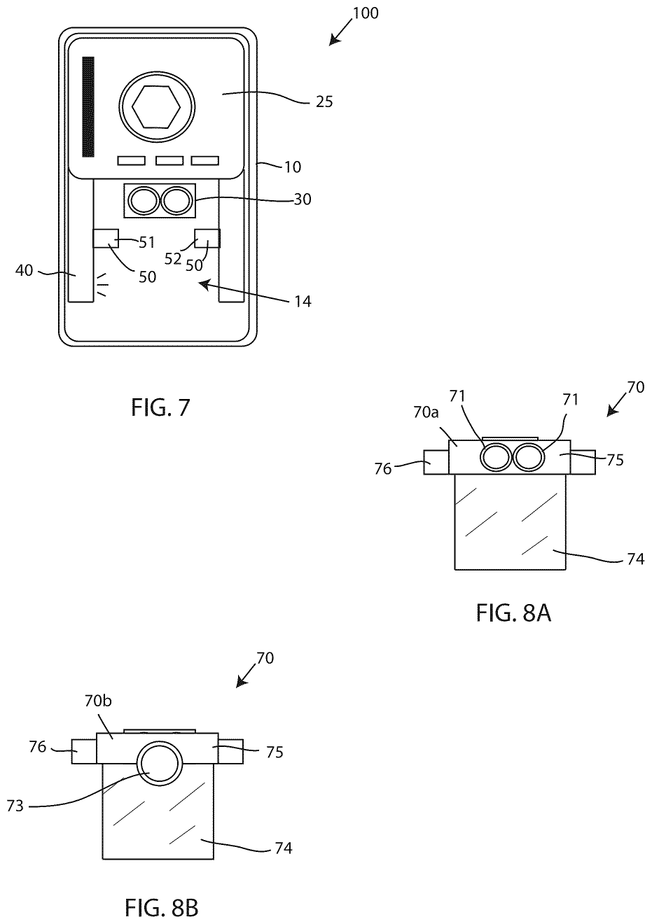

FIG. 9 depicts a schematic view of the pod 100 with an accessory inserted therein, in accordance with embodiments of the present invention. In the illustrated embodiment, the pod 100 is shown with accessory 70 disposed within the pod 100. As can be seen in FIG. 9, the accessory 70 resides within the interior region 14 of the casing 10 when inserted into the pod 100, and the pod engagement mechanism 76 of the accessory 70 engages the accessory receiving mechanism 50 of the pod 100 to removably secure the accessory 70 within the pod 100. The fluidic coupling of the accessory 70 is fluidically coupled/mated with the fluidic coupling 30 in this position, such that a managed flow of gas from the flow management device of the pod 100 can flow through the accessory 70 and out of the outlet 73, or a flow of gas can flow into the outlet 73 through accessory 70 and flow management device of the pod 100 to a source (e.g. suction operation). Tubing is connected to the outlet 73 for delivery to the patient.

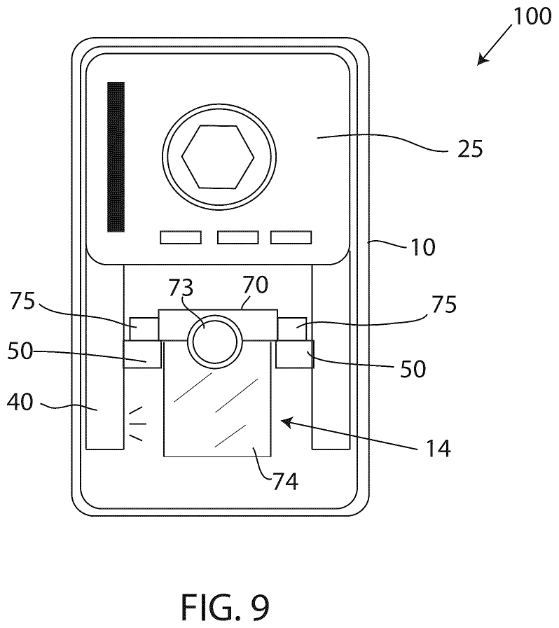

The sensor 40 is in a position to monitor the canister 74 of the accessory 70 when the accessory 70 is inserted within the pod 100. The sensor 40 can detect a current state of the accessory 70, such as a current fluid level within the canister 74, and report the current state to the control interface 25. The sensor 40 can detect a change in a state of the accessory 70, for example, from a first state to a second state. If a fluid level increases or decreases from a baseline fluid level, or if the fluid level rises or falls below a predetermined level within the canister 74, the sensor 40 transmits a signal to the control interface 25 to indicate a change in state. In a first example, embodied by FIGS. 10A-10B, the first state of the removable accessory 70 is a level of a medium inside a canister 74 of the removable accessory 70 that is below a predefined level 45 shown by dotted line in FIG. 10A. In this example, it is acceptable to have a level of medium anywhere below the predefined level 45. As medium is added to the canister 74, for example, via suction from external sources and added into the canister, the level of medium rises. The sensor 40 is configured to detect when the medium rises above the predefined level 45, which is defined as the second state of the removable accessory, as shown in FIG. 10B. In a second example, embodied by FIGS. 10C-10D, the first state of the removable accessory 70 is a level of a medium inside a canister 74 of the removable accessory 70 that is above a predefined level 45 shown by dotted line in FIG. 10C. In this example, it is acceptable to have a level of medium anywhere above the predefined level 45. As medium is depleted, for example by evaporation, the canister 74, the level of medium lowers. The sensor 40 is configured to detect when the medium falls below the predefined level 45, which is defined as the second state of the removable accessory 70, as shown in FIG. 10D.

The sensor 40 can detect the fluid level by emitting light towards the canister 74. The medium within the canister 74 reflects the light back toward the sensor 40. The sensor 40 measures the light reflected back toward the sensor 40 from the medium within the canister 74, and transforms the measurement into an electrical signal for processing by the control interface 25, or in some embodiments, a processor dedicated to the sensor 40 and/or positioned within the sensor housing. Alternatively, the sensor 40 can detect the fluid level by a transmitter located on one side of the pod casing 10 emitting a light beam through the canister 74 towards a receiver located on the oppose side of the pod casing 10. The medium within the canister 74 interrupts the light so that the interrupted light does not reach the receiver of sensor on the other side. The sensor 40 measures the light received and/or not received and transforms the measurement into an electrical signal for processing by the control interface 25, or in some embodiments, a processor dedicated to the sensor 40 and/or positioned within the sensor housing.

When the sensor 40 detects the change from the first state to the second state, the sensor 40 sends a signal to the control interface 25 of the pod 100 for performing at least one function. The at least one function can include notifying a clinician with at least one of an audible notification, a visual notification on a graphical user interface, an indicator, a transmission to a remote computer, or a combination thereof.

The audible notification may be an alarm that makes a noise through a speaker electrically coupled to the control interface 25; the speaker can be a built-in speaker of the pod 100. The purpose of the alarm is to alert a clinician to the need to act with respect to a specific accessory 70 disposed within a specific pod 100. In practice, the clinician will hear the alarm and follow training and appropriate rules and regulations for either replacing the removable accessory 70, replenishing the accessory with the proper medium, disposing the medium contained within the canister 74, etc.

The visual notification may be a light, LED, or graphic that activates on a graphical user interface associated with the pod 100 either built-in with the pod 100 or located proximate the pod 100, that emits a light conspicuous enough to be noticed by a clinician. The purpose of the visual notification is to alert a clinician to the need to act with respect to a specific accessory 70 disposed within a specific pod 100. In practice, the clinician will see the visual notification and follow training and appropriate rules and regulations for either replacing the removable accessory 70, replenishing the accessory with the proper medium, disposing the medium contained within the canister 74, etc.

The indicator may be a light bulb or LED located within the hospital room, on the patient bed, computer stations external to the patient's room, wirelessly connected or wired to the control interface 25 of the pod 100, configured to emit a light conspicuous enough to be noticed by a clinician. The purpose of indicator is to alert a clinician to the need to act with respect to a specific accessory 70 disposed within a specific pod 100. In practice, the clinician will see the visual notification and follow training and appropriate rules and regulations for either replacing the removable accessory 70, replenishing the accessory with the proper medium, disposing the medium contained within the canister 74, etc.

The transmission to a remote computer may be a signal transmitted over the Internet to a computer, tablet, laptop, etc. being monitored by a clinician. The purpose of the transmission to a remote computer is to alert a clinician to the need to act with respect to a specific accessory 70 disposed within a specific pod 100, even if the clinician is not physically present in the patient room, near the patient's room, or not at a dedicated clinician station. In practice, the clinician will see the transmission and follow training and appropriate rules and regulations for either replacing the removable accessory 70, replenishing the accessory with the proper medium, disposing the medium contained within the canister 74, etc.

The control interface 25 of the pod 100 can be programmed, for example, via user interface, to trigger one type of notification or a combination of types of notifications to work in concert in alerting a clinician. The control interface 25 can trigger the audible notification, the visual notification on a graphical user interface, the indicator, and/or the transmission to the remote computer, in response to receiving the signal from the sensor 40.

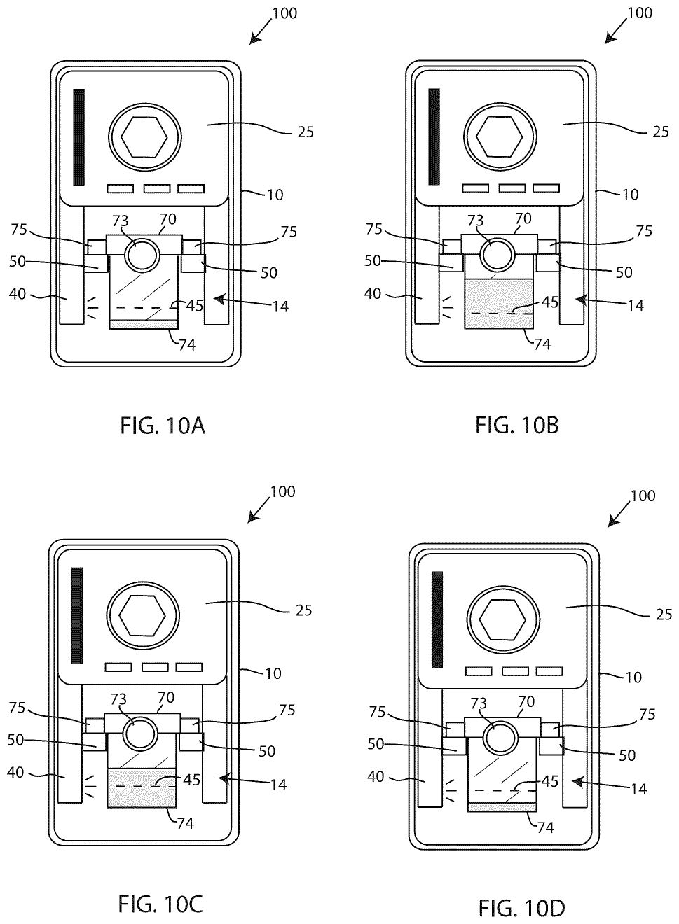

FIG. 11 depicts a schematic view of the pod 100 with an accessory inserted therein and a cover covering the accessory, in accordance with embodiments of the present invention. In the illustrated embodiment, the pod 100 includes a cover 60 that is configured to cover at least a portion of the inserted accessory 70. The cover 60 is optionally used to prevent incidental contact with the accessory 70, deter non-clinicians from removing the accessory 70, hide contents of the accessory 70, etc. The cover 60 has a center opening for quick visual inspection of whether an accessory is inserted in the pod 100 or not. The cover 60 can be permanently attached to the casing 10 so that cover 60 (e.g. via a hinge connection) or the cover 60 can be non-permanently attached to the casing 10, configured to be snapped into place and detached from the casing 10.

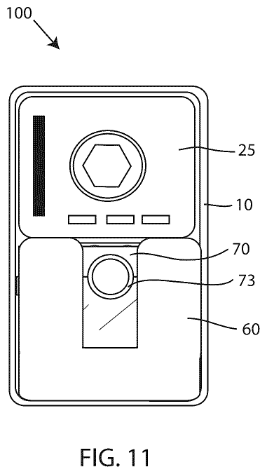

FIG. 12 shows a pod system 1000 incorporated into various objects in an environment, according to embodiments of the present invention. The pod system 1000 includes a plurality of pods 100 having flow management devices 20 that are at least partially recessed within finished surface 64. The finished surface 64 can be a wall 65 of a room, a headwall 62 of a bed unit, an exterior surface of a freestanding gas delivery system 63, a ceiling column, a boom, and/or an exterior surface of a boom 61 of a gas delivery system. Fluidic connections between each pod 100 and the rough-in fluidic connections of the gas sources are located behind finished surface 64. Because the pod system 1000 is recessed within the finished surface 64, the SOT devices do not protrude from the finished surface 64 and accessibility is limited. As a result, the pod system 1000, and the pods therein, are largely protected from damage from incidental contact, clinicians and non-clinicians cannot easily remove the SOT devices from the wall in a hospital room to use in another room, and the fluidic connections to the gas sources are protected and located behind the finished surface 64 so that accidental disconnection leading to unintentional gas flow into the room cannot happen. The pod system 1000 includes a receptacle recessed within finished surface 64, such as rough-in assembly 7, or a receptacle designed as several rough-in assemblies as one unit. Moreover, the pod system 1000 includes the removable accessories configured to be fluidically coupled to the plurality of pods. Fluidic connections between each removable accessory and each pod are located within the pod and behind finished surface 64. A power source is electrically coupled to a control interface of each pod of the pod system 1000 for controlling a flow through the pods 100; the power source may include one or more electrical wires running from a remote breaker behind the finished surface 64.

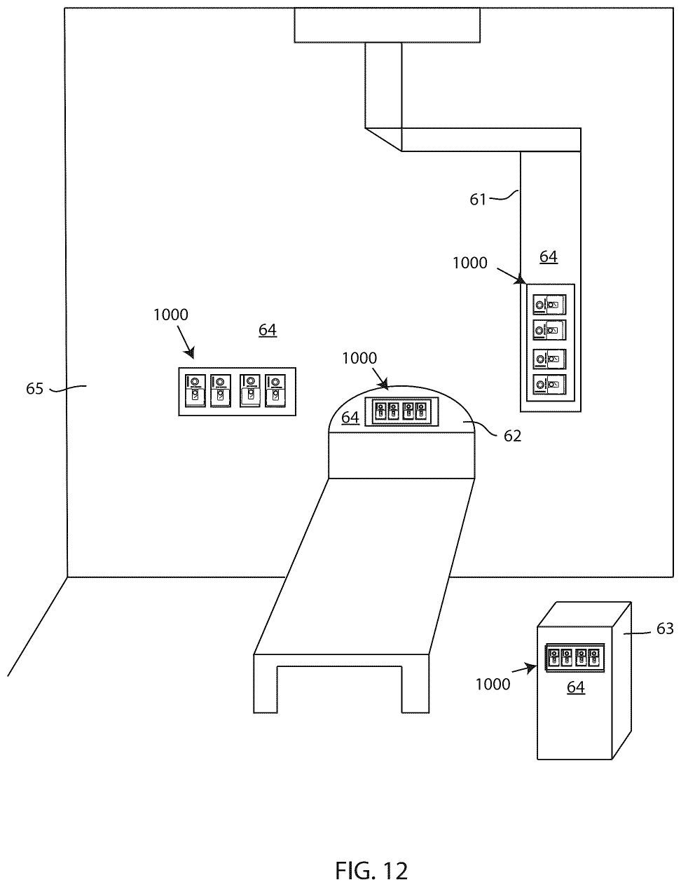

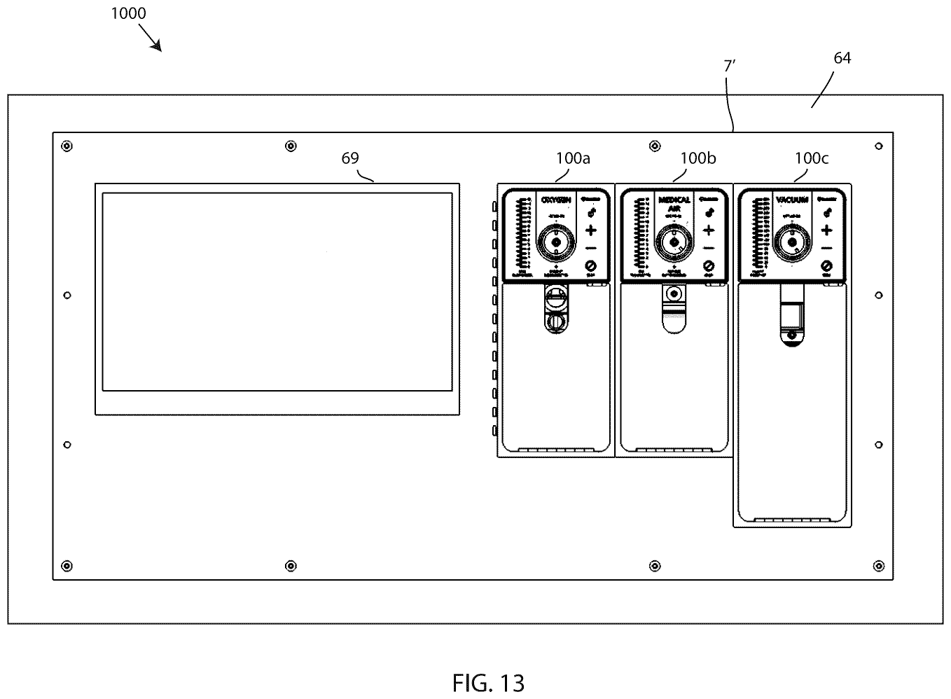

FIGS. 13-14 depict a pod system 1000 according to an exemplary embodiment of the present invention. The pod system 1000 illustrated in FIG. 13 includes a rough-in assembly cover 7′ designed with openings to accommodate three pods 100a, 100b, 100c and a display 69 for displaying various patient-related information, room information, pod and pod system information, location information, etc. The display 69 is also used to communicate notifications generated by the control interface 25 to indicate that the sensor detected a change in state of the accessory, and an action(s) needs to be taken. The rough-in assembly cover 7′ is installed onto the finished surface 64 and cooperates with/is attached to rough-in assembly 7 installed behind the finished surface 64 that supports the fluidic connections to the sources 7′. The rough-in assembly cover 7′ may be attached to rough-in assembly 7 behind the wall prior to of after the pods 100a, 100b, 100c are removably inserted into the rough-in assembly. As described above, each pod is specific to a type of gas associated with a source. For example, pod 100a is specifically designed for managing a flow of gas associated with a first source, pod 100b is specifically designed for managing a flow of gas associated with a second source, and pod 100c is specifically designed for managing a flow of gas associated with a third source. Each pod 100a. 100b, 100c is removably inserted such that each pod 100a, 100b, 100c is substantially recessed within or otherwise located behind the finished surface 64.

The flow management devices of the pods 100a, 100b, 100c and accessories installed within the pods 100a, 100b, 100c are located behind the finished surface 64. A depth of the casings 10a, 10b, 10c can be sized and dimensioned to accommodate the flow management devices 20 of the pods 100a, 100b, 100c, and the accessories 70a, 70b, 70c removably inserted into the pod 100a, 100b, 100c, respectively. The casings 10a, 10b, 10c extend into the rough-in assembly 7 installed behind the finished surface 64, which allows for the flow management devices 20 and the accessories 70a, 70b, 70c to be supported behind the finished surface 64 of a hospital room wall, for example. FIG. 14 is a perspective view of pod system 1000 shown in FIG. 13, with a cover 60 installed onto the pods, in accordance with embodiments of the present invention. The cover 60 is optionally removably installed over the accessories to prevent incidental contact with the accessory 70, deter non-clinicians from removing the accessory 70, hide contents of the accessory 70, etc.

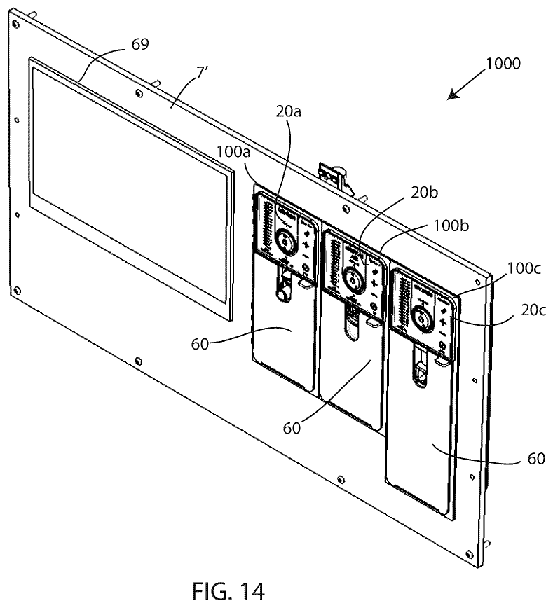

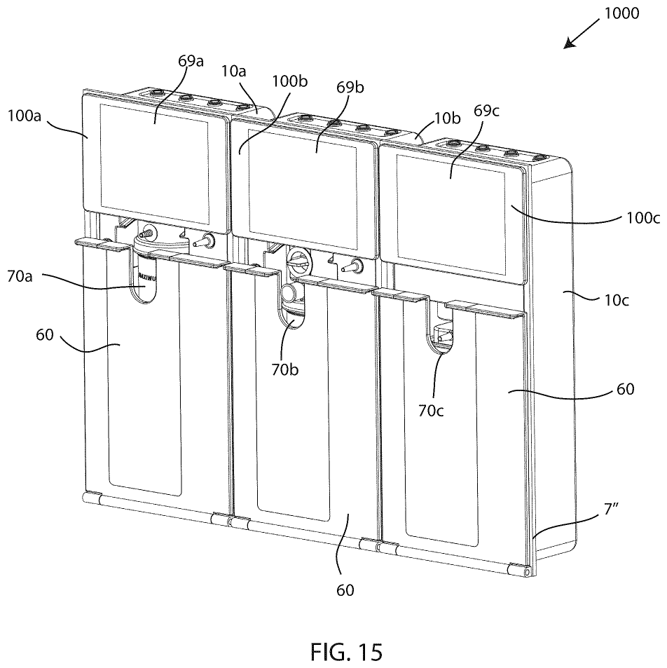

FIGS. 15-16 depict another embodiment of a pod system 1000. The pod system 1000 illustrated in FIG. 15 includes a rough-in assembly cover 7″ designed with openings to accommodate three pods 100a, 100b, 100c and, unlike the embodiment shown in FIGS. 13-14, a display 69a, 69b, 69c disposed on a front side of the pod 100a, 100b, 100c instead of a single display 69 to the side of the pods, for displaying various patient-related information, room information, pod and pod system information, location information, etc. The displays 69a, 69b, 69c can be used to display notifications triggered by the sensor 20. The rough-in assembly cover 7″ is installed onto the finished surface and cooperates with/is attached to rough-in assembly 7 installed behind the finished surface 64 that supports the fluidic connections to the sources. The rough-in assembly cover 7″ may be attached to rough-in assembly 7 behind the wall prior to or after the pods 100a, 100b, 100c are removably inserted into the rough-in assembly 7. As described above, each pod is specific to a type of gas associated with a source; more than one pod may contain the same type of gas, or each pod is associated with a different type of gas. For example, pod 100a is specifically designed for managing a flow of gas associated with a first source, pod 100b is specifically designed for managing a flow of gas associated with a second source, and pod 100c is specifically designed for managing a flow of gas associated with a third source. Each pod 100a. 100b, 100c is removably inserted such that each pod 100a, 100b, 100c is substantially recessed within or otherwise located behind the finished surface.

As shown in FIGS. 15 and 16, the flow management devices of the pods 100a, 100b, 100c and accessories installed within the pods 100a, 100b, 100c are located behind the finished surface when the rough-in assembly cover 7″ is placed over the rough-in assembly 7 located behind the wall. FIG. 15 is a perspective view of pod system 1000 and FIG. 16 is a front view of the pod system 1000, in accordance with embodiments of the present invention. A depth of the casings 10a, 10b, 10c of the pods 100a, 100b, 100c is depicted in FIG. 15. The depth of the casings 10a, 10b, 10c can be sized and dimensioned to accommodate the flow management devices 20 of the pods 100a, 100b, 100c, and the accessories 70a, 70b, 70c removably inserted into the pod 100a, 100b, 100c, respectively. The casings 10a, 10b, 10c extend into the rough-in assembly 7 installed behind the finished surface, which allows for the flow management devices 20 and the accessories 70a, 70b, 70c to be supported behind the finished surface of a hospital room wall, for example. A cover 60 is installed onto the pods, which is optionally removably installed over the accessories to prevent incidental contact with the accessory 70, deter non-clinicians from removing the accessory 70, hide contents of the accessory 70, etc.

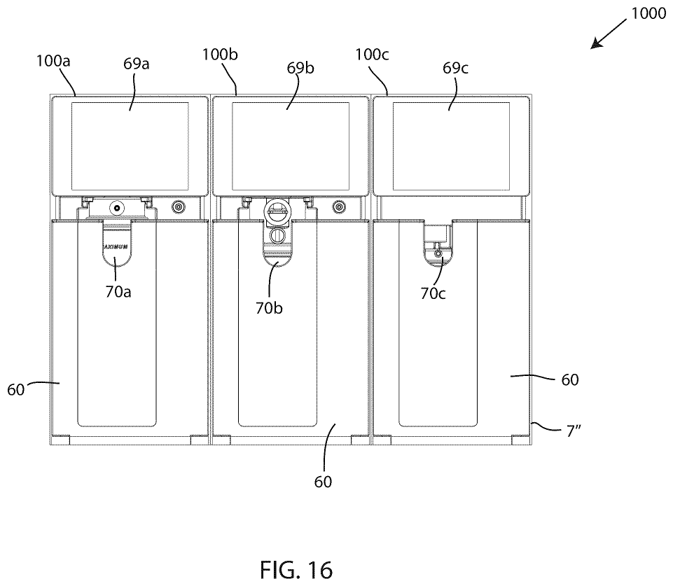

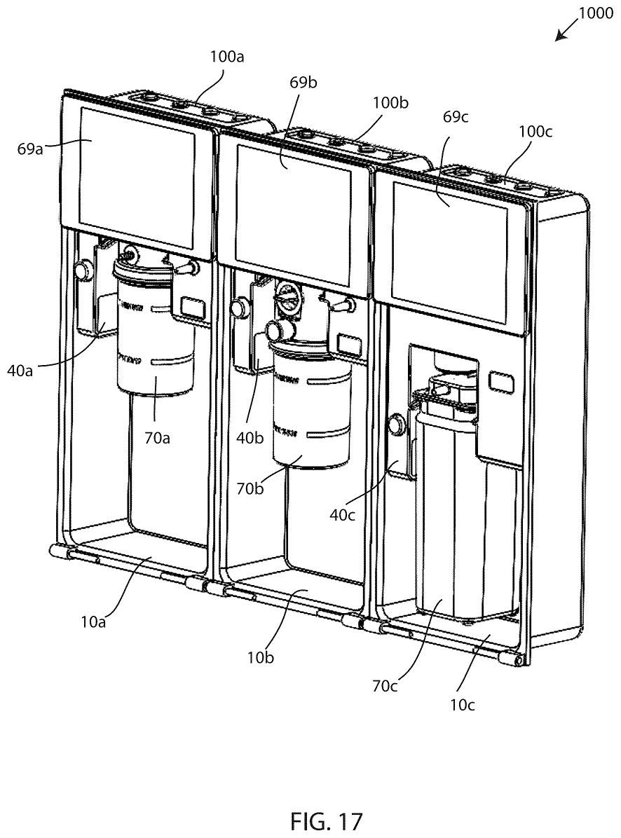

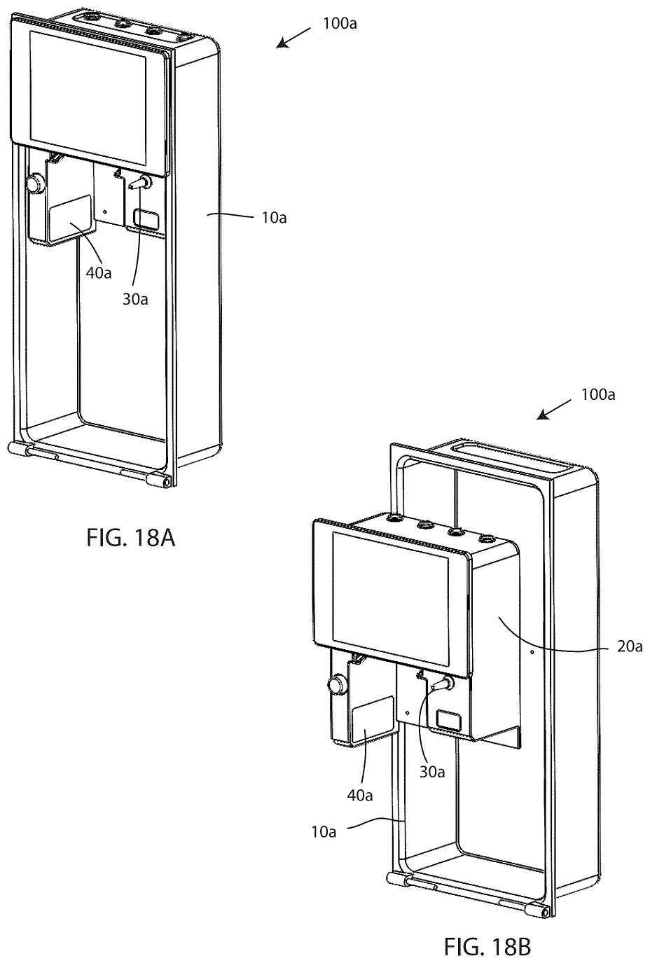

With continued reference to the drawings, FIG. 17 depicts pods 100a, 100b, 100c, according to an exemplary embodiment of the present invention. In the embodiment illustrated in FIG. 17, the pods 100a, 100b, 100c include a flow management device and at least one fluidic coupling within the pod casing 10a, 10b, 10c that is fluidically connected to the flow management device, as described above. The casings include one or more knockouts to facilitate fluidic connections with fluid sources located remotely from the pods 100a, 100b, 100c. The at least one fluidic coupling is configured to fluidically connect with a removable accessory 70a, 70b, 70c insertable within the pod casing 10a, 10b, 10c. Further, the pods 100a, 100b, 100c each include a sensor 40a, 40b, 40c, respectively. FIG. 18A depicts a perspective view of an embodiment of the pod 100a without an accessory inserted therein, in accordance with embodiments of the present invention. FIG. 18B depicts a perspective view of pod 100a removed from the pod casing 10a. Pod 100a may be a pod specifically for oxygen or medical air delivery to the patient and includes a sensor 40a for detecting a change in a state of an accessory inserted into pod 100a. FIG. 19A depicts a perspective view of an embodiment of the pod 100c without an accessory inserted therein, in accordance with embodiments of the present invention. FIG. 19B depicts a perspective view of pod 100c removed from the pod casing 10c. Pod 100c may be a pod specifically for vacuum applications, and includes sensor 40c for detecting a change in a state of an accessory inserted therein.

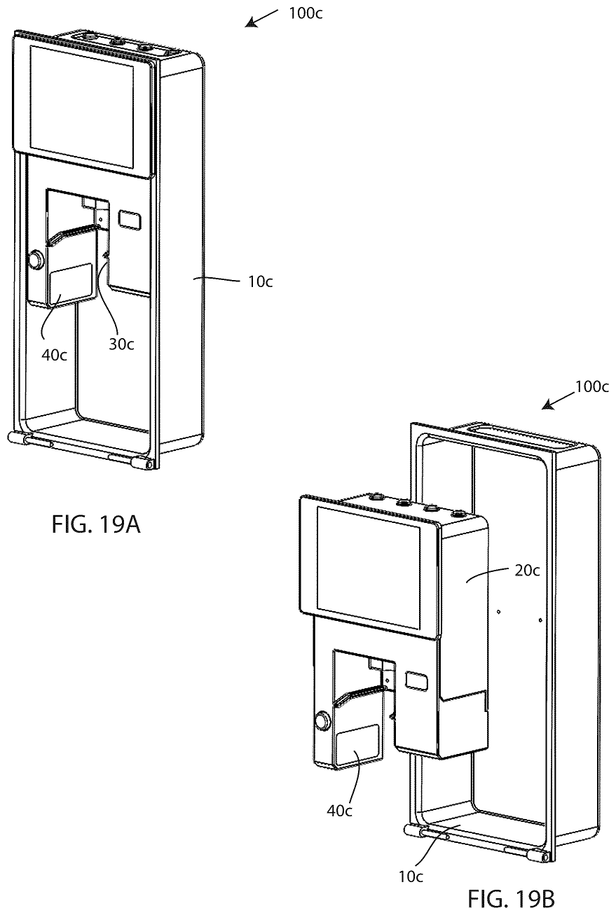

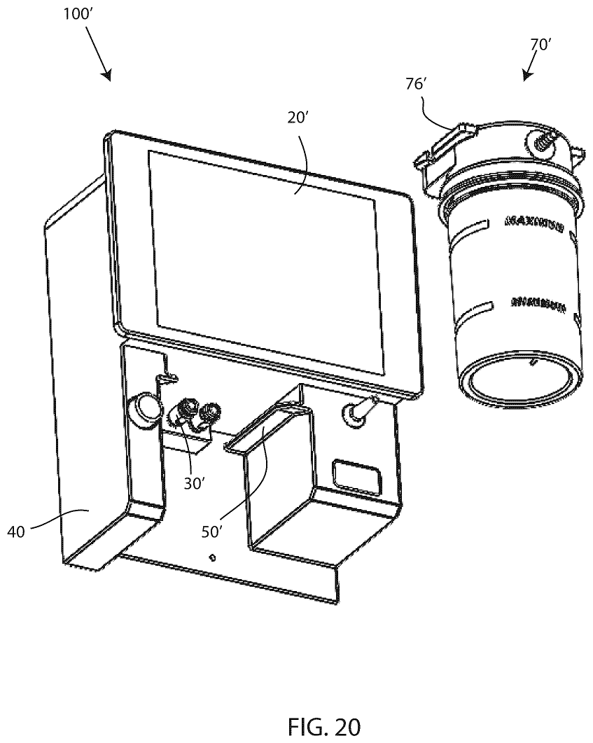

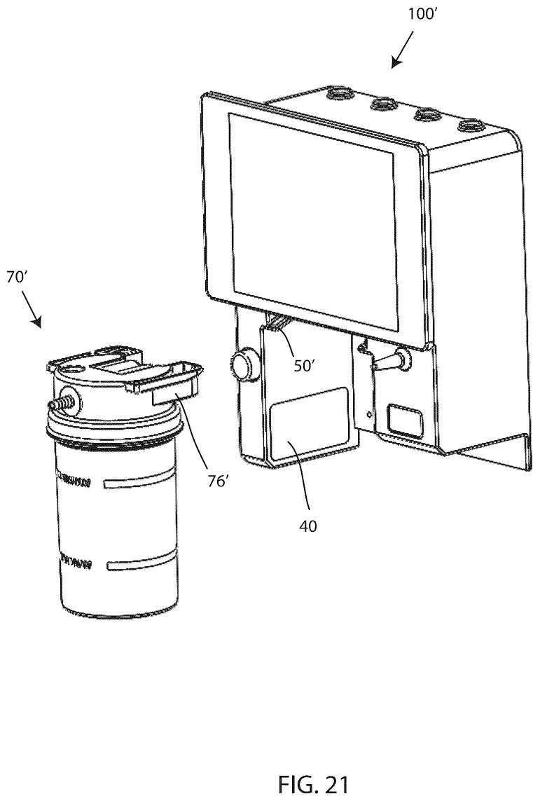

FIGS. 20-22 depict a connection between an accessory 70′ and a pod 100′, in accordance with an exemplary embodiment of the present invention. The accessory 70′ is configured to fluidically and mechanically connect to the fluidic coupling 30′ built into the pod 100′ so that the gas flows through the accessory 70′ and out to the patient. One accessory is insertable into the pod 100′ at a given time, but there are many different accessories that can be used with pod 100′. The accessory 70′ resides within the pod casing (not shown in FIGS. 20-22) when inserted or otherwise attached to the pod 100′, and is removable from the pod 100′ so that a new accessory can be inserted into the pod 100′. Underneath the flow management device 20′ resides accessory receiving mechanism 50′. The accessory receiving mechanism 50′ facilitates an insertion and fluidic coupling of the accessory 70′. The accessory receiving mechanism 50′ includes a first receiving structure 51′ and a second receiving structure 52′ on a second side opposite the first side. The first receiving structure 51′ and the second receiving structure 52′ extend towards the rear wall of the casing, starting proximate or at the front of the casing. In the illustrated embodiment shown in FIGS. 20-22, the accessory receiving mechanism 50′ includes a ramped surface proximate the front side of the pod 100 that initially makes contact with and helps guides the pod engagement mechanism 76′ of accessory 70′ into the casing and into proper mating position with the fluidic coupling 30′ of the pod 100′. The ramped surface flattens out to support, at least partially, the weight of the accessory 70′ when inserted and mated within the pod 100′. The accessory 70′ can be driven up the ramped surface and towards the back of the pod 100′ by a user, guided by the mechanical interaction between the accessory receiving mechanism 50′ and the pod engagement mechanism 76′. With continued driving force, the accessory 70′ moves to a fully inserted position and mates with the fluidic coupling 30′ as a function of the driving force applied the accessory 70′. FIG. 21 depicts the accessory 70′ and the pod 100′ prior to being inserted into the pod 100′ and FIG. 22 depicts the accessory 70′ fully inserted within the pod 100′ such that the accessory 70′ is in fluidic communication with the flow management device 20′ by way of the fluidic coupling 30′, and the sensor 40 is proximate to the accessory 70′.

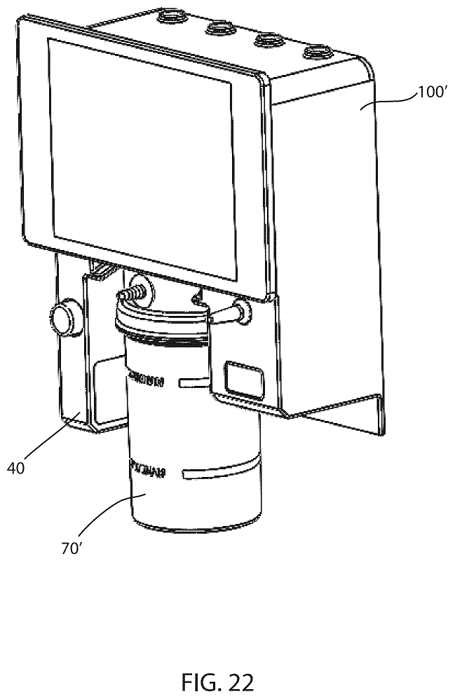

As described above, embodiments of the pod system 1000 are compatible with many different types of removable accessories that are each capable of being inserted into a pod. FIGS. 23-25 depict example accessories that can be used with the pod system 100. FIG. 23 depicts accessory 701 which is a nebulizer used in conjunction with the flow of oxygen managed by a pod of pod system 100. The removable accessory 701 includes a body portion 75 that is a lid or cover having a coupling 71 and an outlet 73. The accessory 701 includes a pod engagement mechanism 76 disposed on the body portion 75 for proper insertion and connection to a pod. The pod engagement mechanism76 includes a first engagement structure at a first location on the body portion 75 and a second engagement structure at a second location of the body portion, opposite the first location. The first engagement structure and the second engagement structure of the pod engagement mechanism 76 is a protrusion or other lip or extension of the body portion 75 that is configured to cooperate with an accessory receiving mechanism of the pod. The accessory 701 includes incudes a canister 74 removably attached (e.g. threaded) to the body portion which functions as a container or storage device that has an interior space that allows a flow of oxygen (or other gas) between the fluidic coupling 71 that mates with the fluidic coupling of the pod and the outlet 73. The interior space of the canister 74 may store a fluid, such as water, for humidifying the oxygen flowing through the accessory 701.

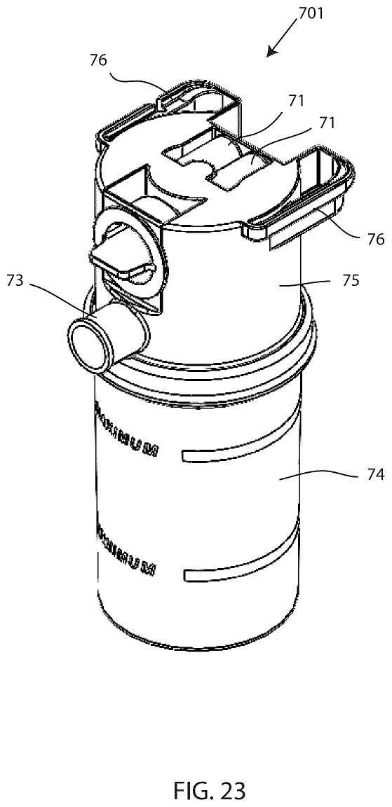

FIG. 24 depicts accessory 702 which is a humidifier used in conjunction with the flow of medical air managed by a pod of pod system 100. The removable accessory 702 includes a body portion 75 that is a lid or cover having a coupling 71 and an outlet 73. The accessory 702 includes a pod engagement mechanism 76 disposed on the body portion 75 for proper insertion and connection to a pod. The pod engagement mechanism76 includes a first engagement structure at a first location on the body portion 75 and a second engagement structure at a second location of the body portion, opposite the first location. The first engagement structure and the second engagement structure of the pod engagement mechanism 76 is a protrusion or other lip or extension of the body portion 75 that is configured to cooperate with an accessory receiving mechanism of the pod. The accessory 702 includes incudes a canister 74 removably attached (e.g. threaded) to the body portion which functions as a container or storage device that has an interior space that allows a flow of medical air (or other gas) between the fluidic coupling 71 that mates with the fluidic coupling of the pod and the outlet 73. The interior space of the canister 74 may store a fluid, such as water, for humidifying the medical air flowing through the accessory 701.

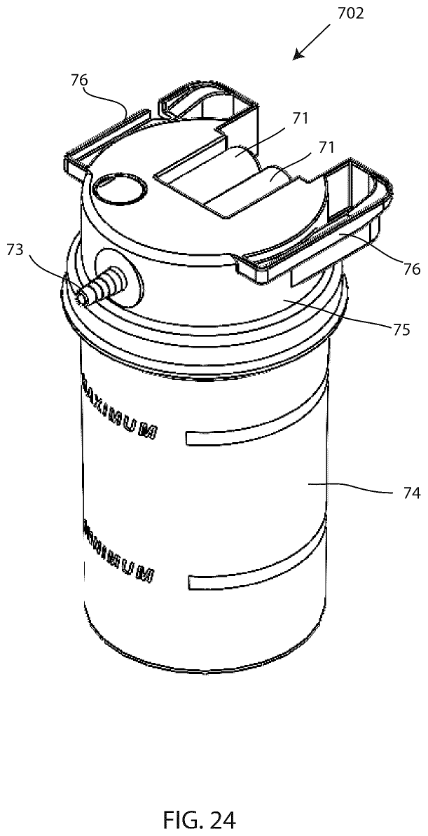

FIG. 25 depicts accessory 703 which is a vacuum container used in conjunction with a vacuum managed by a pod of pod system 100. The removable accessory 703 includes a body portion 75 that is a lid or cover having a coupling 71 and an outlet 73. The accessory 703 includes a pod engagement mechanism 76 disposed on the body portion 75 for proper insertion and connection to a pod. The pod engagement mechanism 76 is a protrusion or other lip or extension of the body portion 75 that is configured to cooperate with an accessory receiving mechanism of the pod. The accessory 703 includes incudes a canister 74 removably attached (e.g. threaded) to the body portion which functions as a container or storage device that has an interior space that allows for storage of fluid and biological material drawn into the accessory 703 via the suction force into the accessory 703.

In alternative embodiments of the present invention, the sensor can be a mechanical sensor, such as a float, a float switch, a water level float sensor, a continuous level sensor, a float level switch, and the like, positioned within the accessory. The mechanical sensor includes a float that rises and falls depending on the level of the water, or other fluid, within the accessory. If the float rises past a certain point, a signal is sent to the controller to activate an alarm or automatic response. Conversely, if the float falls past a certain point, a signal is sent to the controller to activate an alarm or an automatic response. The mechanical sensor is communicatively coupled to the control interface 25, for example, a wire having a connector that mates with the control interface 25 to establish an electrical connection between the mechanical sensor and the control interface 25. The wire can extend through an opening in the accessory and can be connected by an installer or clinician when the accessory is inserted within the pod.

In further embodiments of the invention, in lieu of or in addition to an alarm or other notification, one or more functions can be performed in response to receiving a signal from the sensor 40. Automatic responses that limit or wholly avoid clinician involvement can be performed by the control interface 25 in response to the sensor 40 detecting a change. For example, the control interface 25, in response to the sensor signal, may evacuate the canister 74 of the removable accessory 70c to remove a medium from the canister 74, return the removable accessory 70a to the first state by filling the canister 74 with the required medium, injecting a solution into a canister 74 of the removable accessory storing a medium, and the like.

In embodiments of the pod system 1000 having at least one pod (e.g. pod 100c) dedicated to a vacuum function with a complimentary accessory (e.g. accessory 703) for vacuum applications, fluid or biological material stored within the inserted accessory can be automatically evacuated directly from the canister of the accessory to a collection vessel located remote from the hospital room, in response to the sensor 40 detecting that the fluid or biological material has exceeded a maximum fill level within the canister. Conventional suction regulators are connected to existing ports located on the wall of the room and include a canister for collection of fluids from the patient. When the canister fills up, clinicians manually detach the canister and discard the canister in a biohazard bag for disposal. Canister removal may occur multiple times during a patient stay and must be removed after the patient stay.

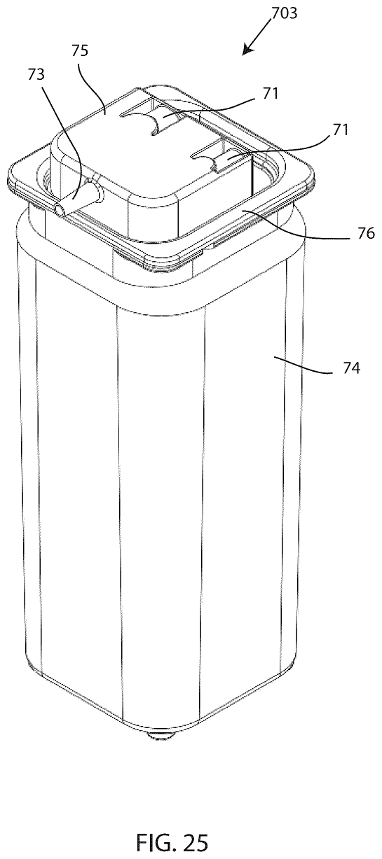

Embodiments of the pod system 1000 can be used to evacuate the fluids from the accessory to a collection vessel remote from the hospital room without requiring the canister to be detached because the vacuum pod of the pod system 1000 is located behind a finished surface. For instance, when the sensor 40 senses that the fluid in the canister has reached or exceeded the maximum predefined limit, the control interface 25 can actuate pump 80 and potentially one or more valves to evacuate the fluid from the canister to a collection vessel 90 remote from the hospital room and/or a main plumbing system of a hospital. A discharge line can be plumbed into the rough-assembly 7 proximate the vacuum pod (e.g. pod 100c) to fluidically connect to the interior of the vacuum pod accessory such that the fluids can be evacuated from the accessory directly to the remote collection vessel or main plumbing system without requiring the canister to be removed from the pod and without requiring clinician involvement. FIG. 26 depicts a schematic view of a fluid evacuation system 500. The pod system 1000 is connected to a collection vessel 90 or main plumbing system design to safely store or dispose of biological waste and fluids drawn from a patient in a hospital room during usage of a suction regulator of pod system 1000. The accessory 70c of pod 100c is fluidically connected to the collection vessel or main plumbing system via discharge line 95; pump 80 is disposed between the pod system 1000 and the collection vessel/main plumbing system along discharge line 95.

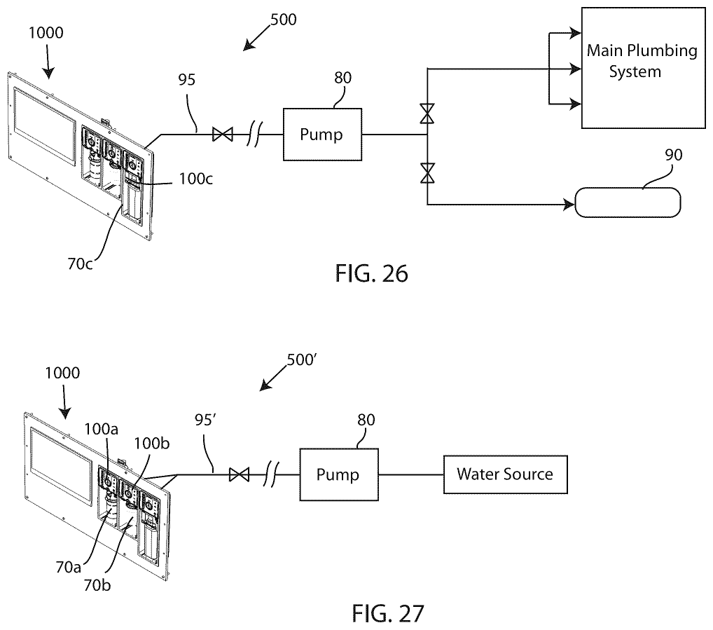

In embodiments of the pod system 1000 having at least one pod (e.g. pod 100a, 100b) dedicated to delivering medical air or oxygen with a complimentary accessory (e.g. accessory 701, 702) for humidifying the air/oxygen prior to delivery to patient, the water in the accessory can be refilled, in response to the sensor 40 detecting that water has exceeded a minimum fill level within the canister. Embodiments of the pod system 1000 can be used to add water to the accessory from a remote water source without requiring a clinician to manually refill the canister. For instance, when the sensor 40 senses that the water in the canister has fallen below or reaches the maximum predefined limit, the control interface 25 can actuate pump 80 and potentially one or more valves to evacuate the fluid from the canister to a collection vessel 90 remote from the hospital room and/or a main plumbing system of a hospital. A discharge line can be plumbed into the rough-assembly 7 proximate the pod (e.g. pod 100a, 100b) to fluidically connect to the interior of the vacuum pod accessory such that the fluids can be added to the accessory without requiring the canister to be removed from the pod and without requiring clinician involvement. FIG. 27 depicts a schematic view of a fluid filling system 500′. The pod system 1000 is connected to a water source. The accessory 70a, 70b of pod 100a, 100b is fluidically connected to the water source via line 95′.

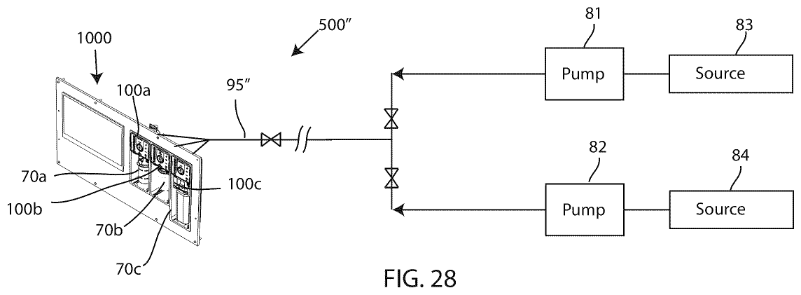

In embodiments of the pod system 1000 having pods 100a, 100b, 100c dedicated to delivering medical air or oxygen, vacuum functions, etc. with a complimentary accessories 70a, 70b, 70c, various solutions can be injected into the canister, in response to the sensor 40 detecting a need for an additional component, such as a solidifier. Embodiments of the pod system 1000 can be used to add a solution to any one of the accessories from remote sources 83, 84 without requiring a clinician to manually add the solution to the canister. For instance, when the control interface receives the signal from the sensor 40, the control interface 25 can actuate one or both pumps 81, 82 and potentially one or more valves to inject one or more solutions into the accessories. Multiple lines can be plumbed into the rough-assembly 7 proximate the pod (e.g. pod 100a, 100b, 100c) to fluidically connect to the interior of the pod accessory such that the fluids can be added to the accessory without requiring the canister to be removed from the pod and without requiring clinician involvement. FIG. 28 depicts a schematic view of a fluid filling system 500″. The pod system 1000 is connected to a plurality of sources 83, 84 (two sources shown but could be additional sources). The accessory 70a, 70b of pod 100a, 100b is fluidically connected to the water source via line 95″. Pump 81 can operate to inject to inject a solution from source 83 into the interior of the canister in response to a signal from the control interface 25 based on the sensor 40 detecting the change. Pump 82 can operate to inject to inject a different solution from source 84 into the interior of the canister in response to a signal from the control interface 25 based on the sensor 40 detecting the change. Embodiments of the solution added to the accessories and stored in sources 83, 84 include a solidifier which turns waste into a gel-like state to eliminate spills and splashes and prepare the waste for safe disposal as required. Another solution can be water.

While this disclosure has been described in conjunction with the specific embodiments outlined above, it is evident that many alternatives, modifications and variations will be apparent to those skilled in the art. Accordingly, the preferred embodiments of the present disclosure as set forth above are intended to be illustrative, not limiting. Various changes may be made without departing from the spirit and scope of the invention, as required by the following claims. The claims provide the scope of the coverage of the invention and should not be limited to the specific examples provided herein.

What is claimed is:

1. A pod comprising:
a flow management device disposed within a pod casing;
at least one fluidic coupling within the casing that is fluidically connected to the flow management device, the at least one fluidic coupling configured to fluidically connect with a removable accessory insertable within the pod casing; and
a sensor configured to detect a change from a first state to a second state of the removable accessory.

2. The pod of claim 1, wherein the first state of the removable accessory is a level of a medium inside a canister of the removable accessory that is below a predefined level, and the second state of the removable accessory is the level of the medium inside the canister exceeding the predefined level.

3. The pod of claim 1, wherein the first state of the removable accessory is a level of a medium inside a canister of the removable accessory that is above a predefined level, and the second state of the canister is the level of the medium inside the canister falling below the predefined level.

4. The pod of claim 1, wherein, when the sensor detects the change from the first state to the second state, the sensor sends a signal to a control interface of the pod for performing at least one function.

5. The pod of claim 4, wherein the at least one function is returning the removable accessory to the first state.

6. The pod of claim 4, wherein the at least one function is an injection of a solution into a canister of the removable accessory storing a medium.

7. The pod of claim 4, wherein the at least one function is filling a canister of the removable accessory with an additional medium.

8. The pod of claim 4, wherein the at least one function is notifying a clinician with at least one of an audible notification, a visual notification on a graphical user interface, an indicator, a transmission to a remote computer, or a combination thereof.

9. The pod of claim 4, wherein the at least one function is an evacuation of a canister of the removable accessory to remove a medium from the canister.

10. The pod of claim 1, wherein the sensor is an optical sensor housed within the pod.

11. The pod according to claim 1, wherein, when the removable accessory is inserted within the pod casing, the at least one fluidic coupling mates with at least one corresponding fluidic coupling of the removable accessory within the casing, and fluid managed by the flow management device flows through the removable accessory and in or out of an outlet of the removable accessory that is fluidically connected to the at least one corresponding fluidic coupling.

12. The pod according to claim 1, further comprising an accessory receiving mechanism located within the pod casing, the accessory receiving mechanism facilitating an insertion and fluidic coupling of the accessory within the pod casing.

13. The pod according to claim 1, further comprising a cover that at least partially covers an interior of the pod casing.

14. The pod according to claim 1, wherein the pod casing is configured to be disposed at least partially within a wall cavity.

15. A system comprising:
a pod comprising a flow management device disposed within a pod casing, at least one fluidic coupling within the casing that is fluidically connected to the flow management device, the at least one fluidic coupling configured to fluidically connect with a removable accessory insertable within the pod casing;
a removable accessory for use with the pod, the removable accessory comprising: a body portion having a first side and a second side, at least one fluidic coupling disposed on the body portion, and a canister operably attached to the body portion, configured to store a medium;
a sensor configured to detect a change from a first state to a second state of the canister; and
a control interface electrically connected to the sensor and configured to receive a signal from the sensor indicating the change.

16. The system of claim 15, wherein the first state of the canister is a level of a medium inside the canister that is below a predefined level, and the second state of the removable accessory is the level of the medium inside the canister exceeding the predefined level.

17. The system of claim 16, wherein the first state of the canister is a level of a medium inside the canister that is above a predefined level, and the second state of the canister is the level of the medium inside the canister falling below the predefined level.

18. The system of claim 15, wherein the control interface sends a notification in response to receiving the signal from the sensor.

19. The system of claim 18, wherein the notification is at least one of an audible alarm, a notification on a graphical user interface, a visual indicator, or a combination thereof.

20. The system of claim 15, further comprising a pump fluidically connected to an interior of the canister, the pump electrically connected to the control interface and configured to inject a solution into the interior of the canister in response to a signal from the control interface based on the sensor detecting the change.

21. The system of claim 15, further comprising a vacuum pump fluidically connected to an interior of the canister, the vacuum electrically connected to the control interface and configured to evacuate the interior of the canister in response to a signal from the control interface based on the sensor detecting the change.

22. The system of claim 15, further comprising a pump fluidically connected to an interior of the canister, the pump electrically connected to the control interface and configured to add an additional medium to the interior of the canister in response to a signal from the control interface based on the sensor detecting the change.

23. A method comprising:
monitoring a removable accessory inserted into a pod;
detecting a change from a first state to a second state of the removable accessory, by a sensor of the pod; and
performing a function by a control interface of the pod, in response to the detecting.

24. The method of claim 23, wherein the function is returning the removable accessory to the first state.

25. The method of claim 23, wherein the function is injecting a solution into a canister of the removable accessory storing a medium.

26. The method of claim 23, wherein the function is filling a canister of the removable accessory with an additional medium.

27. The method of claim 23, wherein the function is notifying a clinician with at least one of an audible alarm, a notification on a graphical user interface, a visual indicator, or a combination thereof.

28. The method of claim 23, wherein the function is evacuating a canister of the removable accessory to remove a medium from the canister.
